SEQUENCE LISTING (12 sequences)

SEQ ID NO: 1 - DNA, 53 bp, synthetic construct
  source 1..53: mol_type = other DNA
atgactgaatataaacttgtggtagttggagctggtggcgtaggcaagagtgc

SEQ ID NO: 2 - DNA, 18 bp, synthetic construct
  source 1..18: mol_type = other DNA
gagctggtggcgtaggca

SEQ ID NO: 3 - DNA, 665 bp, synthetic construct
  source 1..665: mol_type = other DNA
atggattacaaggatcacgatggagactataaggatcacgatatagattacaaagacgatgacgataagatggcgccgaagaaaaagcgtaaagttggtattcatgtgttccggcggcgatggcggagcgtccgttccagtgccgtatttgcatgcgtaactttagccaaagcagcgacctgagccgtcacatccgtacccacaccggtgaaaaaccgttcgcgtgcgacatttgcggccgtaagtttgcgcgtagcgattatctgcgtaaacacaccaagatccacaccggtagccagaagccgttccaatgccgtatttgcatgcgcaactttagccgtagcgcggatctgacccgccatatccgtacccacaccggcgagaagccgtttgcgtgcgacatttgcggtcgcaaatttgcgcgtagcgatgcgctggcgcgtcacaccaaaatccacaccggcagccaaaaaccgtttcagtgtcgtatttgcatgcgcaattttagcgacgattggaacctgagccagcatattcgcacccataccggcgagaagccttttgcttgtgacatttgcggtcgcaagtttgcgcgtagcgcgaacctgacccgtcacaccaaaattcacctgcgtcaaaaggatgcggcgcgtggtagcggcgcg

SEQ ID NO: 4 - protein, 222 aa, synthetic construct
MDYKDHDGDYKDHDIDYKDDDDKMAPKKKRKVGIHGVPAAMAERPFQCRICMRNFSQSSDLSRHIRTHTGEKPFACDICGRKFARSDYLRKHTKIHTGSQKPFQCRICMRNFSRSADLTRHIRTHTGEKPFACDICGRKFARSDALARHTKIHTGSQKPFQCRICMRNFSDDWNLSQHIRTHTGEKPFACDICGRKFARSANLTRHTKIHLRQKDAARGSGA

SEQ ID NO: 5 - DNA, 597 bp, synthetic construct
  source 1..597: mol_type = other DNA
atggcgccgaagaaaaagcgtaaagttggtattcatggtgttccggcggcgatggcggagcgtccgttccagtgccgtatttgcatgcgtaactttagccaaagcagcgacctgagccgtcacatccgtacccacaccggtgaaaaaccgttcgcgtgcgacatttgcggccgtaagtttgcgcgtagcgattatctgcgtaaacacaccaagatccacaccggtagccagaagccgttccaatgccgtatttgcatgcgcaactttagccgtagcgcggatctgacccgccatatccgtacccacaccggcgagaagccgtttgcgtgcgacatttgcggtcgcaaatttgcgcgtagcgatgcgctggcgcgtcacaccaaaatccacaccggcagccaaaaaccgtttcagtgtcgtatttgcatgcgcaattttagcgacgattggaacctgagccagcatattcgcacccataccggcgagaagccttttgcttgtgacatttgcggtcgcaagtttgcgcgtagcgcgaacctgacccgtcacaccaaaattcacctgcgtcaaaaggatgcggcgcgtggtagcggcgcg

SEQ ID NO: 6 - protein, 200 aa, synthetic construct
MMAPKKKRKVGIHGVPAAMAERPFQCRICMRNFSQSSDLSRHIRTHTGEKPFACDICGRKFARSDYLRKHTKIHTGSQKPFQCRICMRNFSRSADLTRHIRTHTGEKPFACDICGRKFARSDALARHTKIHTGSQKPFQCRICMRNFSDDWNLSQHIRTHTGEKPFACDICGRKFARSANLTRHTKIHLRQKDAARGSGA

SEQ ID NO: 7 - DNA, 53 bp, synthetic construct
  source 1..53: mol_type = other DNA
atgactgaatataaacttgtggtagttggagctgatggcgtaggcaagagtgc

SEQ ID NO: 8 - DNA, 18 bp, synthetic construct
  source 1..18: mol_type = other DNA
gagctgatggcgtaggca

SEQ ID NO: 9 - DNA, 666 bp, synthetic construct
  source 1..666: mol_type = other DNA
atggattacaaggatcacgatggagactataaggatcacgatatagattacaaagacgatgacgataagatggcgccgaagaaaaagcgtaaagttggtattcatggtgttccggcggcgatggcggagcgtccgttccagtgccgtatttgcatgcgtaactttagccaaagcagcgacctgagccgtcacatccgtacccacaccggtgaaaaaccgttcgcgtgcgacatttgcggccgtaagtttgcgcgtagcgattatctgcgtaaacacaccaagatccacaccggtagccagaagccgttccaatgccgtatttgcatgcgcaactttagccgtagcgcggatctgacccgccatatccgtacccacaccggcgagaagccgtttgcgtgcgacatttgcggtcgcaaatttgcgcgtagcgatgcgctgacccagcacaccaaaatccacaccggcagccaaaaaccgtttcagtgtcgtatttgcatgcgcaattttagcgacgattggaacctgagccaacatattcgcacccataccggcgagaagccttttgcttgtgacatttgcggtcgcaagtttgcgcgtagcgcgaacctgacccgtcacaccaaaattcacctgcgtcagaaggatgcggcgcgtggtagcggcgcg

SEQ ID NO: 10 - protein, 222 aa, synthetic construct
MDYKDHDGDYKDHDIDYKDDDDKMAPKKKRKVGIHGVPAAMAERPFQCRICMRNFSQSSDLSRHIRTHTGEKPFACDICGRKFARSDYLRKHTKIHTGSQKPFQCRICMRNFSRSADLTRHIRTHTGEKPFACDICGRKFARSDALTQHTKIHTGSQKPFQCRICMRNFSDDWNLSQHIRTHTGEKPFACDICGRKFARSANLTRHTKIHLRQKDAARGSGA

SEQ ID NO: 11 - DNA, 597 bp, synthetic construct
  source 1..597: mol_type = other DNA
atggcgccgaagaaaaagcgtaaagttggtattcatggtgttccggcggcgatggcggagcgtccgttccagtgccgtatttgcatgcgtaactttagccaaagcagcgacctgagccgtcacatccgtacccacaccggtgaaaaaccgttcgcgtgcgacatttgcggccgtaagtttgcgcgtagcgattatctgcgtaaacacaccaagatccacaccggtagccagaagccgttccaatgccgtatttgcatgcgcaactttagccgtagcgcggatctgacccgccatatccgtacccacaccggcgagaagccgtttgcgtgcgacatttgcggtcgcaaatttgcgcgtagcgatgcgctgacccagcacaccaaaatccacaccggcagccaaaaaccgtttcagtgtcgtatttgcatgcgcaattttagcgacgattggaacctgagccaacatattcgcacccataccggcgagaagccttttgcttgtgacatttgcggtcgcaagtttgcgcgtagcgcgaacctgacccgtcacaccaaaattcacctgcgtcagaaggatgcggcgcgtggtagcggcgcg

SEQ ID NO: 12 - protein, 200 aa, synthetic construct
MMAPKKKRKVGIHGVPAAMAERPFQCRICMRNFSQSSDLSRHIRTHTGEKPFACDICGRKFARSDYLRKHTKIHTGSQKPFQCRICMRNFSRSADLTRHIRTHTGEKPFACDICGRKFARSDALTQHTKIHTGSQKPFQCRICMRNFSDDWNLSQHIRTHTGEKPFACDICGRKFARSANLTRHTKIHLRQKDAARGSGA

(19) United States
(12) Patent Application Publication
(10) Pub. No.: US 2025/0051406 A1
(43) Pub. Date: Feb. 13, 2025

(54) CELL TARGETING COMPOSITIONS AND METHODS

(71) Applicant: The Trustees of Indiana University, Bloomington, IN (US)

(72) Inventors: Milan Radovich, Highland Village, TX (US); Jeffrey Solzak, Lantana, TX (US); Chao Wang, Gilbert, AZ (US)

(21) Appl. No.: 18/720,033

(22) PCT Filed: Dec. 15, 2022
(86) PCT No.: PCT/US2022/081619

§ 371 Date: Jun. 14, 2024

Related U.S. Application Data
(60) Provisional application No. 63/289,788, filed on Dec. 15, 2021.

(51) Int. Cl. C07K 14/47; A61K 38/00; G01N 33/53; G01N 33/574

(52) U.S. Cl. CPC C07K 14/4702; G01N 33/5308; G01N 33/57496; A61K 38/00; C07K 2319/33; C07K 2319/43; C07K 2319/81

(57) ABSTRACT

The present disclosure provides cell targeting constructs that specifically target cells of interest through the use of binding agents to cell surface nucleic acids, including without limitation extracellular DNA (exDNA). The cell targeting constructs can be engineered to deliver desired payload, including without limitation therapeutic agents and/or detectable labels, to the target cells.

### FIELD OF THE INVENTION

The general field of the present disclosure is targeting cells. The present disclosure provides a delivery system that can specifically target cells using extracellular nucleic acids attached to the cell surface. Such targeting can be used in diagnostic, therapeutic and other applications.

### BACKGROUND

### Extracellular DNA

Various biological entities are found in bodily fluids, including cells, extracellular microvesicles and cell-free proteins and nucleic acids. Circulating, cell-free, nucleic acids (cfNA) include DNA (cfDNA) and various forms of RNA (cfRNA), including but not limited to messenger RNAs and microRNAs. See, e.g., Pos et al., “Circulating cell-free nucleic acids: characteristics and applications,” Eur J Hum Genet 26:937-945 (2018). The presence of such circulating biomarkers have been used diagnostic, prognostic and theranostic applications.

It was recently reported that extracellular DNA (exDNA) is present on the surface of pancreatic cancer cells and facilitates metastatic behavior. See Wen et al., “Extracellular DNA in Pancreatic Cancer Promotes Cell Invasion and Metastasis,” (2013), Cancer Res; 73(14); 4256-66. These investigators found exDNA associated with pancreatic cancer cells in culture and in tissue sections, but not with normal pancreas cells. The cancer cell-related exDNA appeared to be involved in cell metastatic potential in vitro and DNase I treatment decreased cancer metastasis in an orthotopic xenograft pancreatic cancer mouse model. Id. The investigators conjectured that the exDNA was associated with extracellular traps comprising pancreatic cellular exDNA. Id.

Stressed, damaged and dying cells release intracellular factors into their local microenvironment, including DNA, that activate immune components. Such exDNA can lead to inflammation that contributes to pathogenesis of various autoimmune diseases. exDNA released from cancer cells in response to chemotherapy may activate Toll-like receptor signaling that increases cell survival by inhibiting apoptosis and promoting autophagy. See Anunobi et al., “Extracellular DNA promotes colorectal tumor cell survival after cytotoxic chemotherapy,” (2018) J Surgical Res. 226: pp. 181-191.

### Zinc Finger Proteins (ZFPs)

Zinc finger proteins (ZFPs) are proteins which comprise a zinc finger motif, which is characterized by one or more zinc ions (Zn2+). ZFPs are one of the most abundant groups of proteins and have a wide range of molecular functions. Various ZNFs interact with DNA, RNA, PAR (poly-ADP-ribose) and other proteins and small molecules. ZNFs are implicated in transcriptional regulation, ubiquitin-mediated protein degradation, signal transduction, actin targeting, DNA repair, cell migration, and numerous other processes. Through their ability to regulate gene expression, ZNF proteins participate in numerous physiological processes, including cell proliferation, differentiation, and apoptosis, thereby maintaining tissue homeostasis. See for example Cassandri et al., “Zinc-finger proteins in health and disease,” (2017) Cell Death Discovery 3: p. 17071. The zinc-finger motif is one of the most common DNA-binding motif within eukaryotic transcription factors. The zinc-finger domain binds to its target site by juxtaposing base pairs of the DNA to amino acids in the zinc finger structure. The identity of the amino acids at the contact site define the DNA sequence recognition specificity of the ZFP. Thus, by changing these amino acids, a high degree of selectivity can be achieved toward a given DNA sequence.

Exploiting the zinc-finger recognition mechanism, protein modules containing multiple zinc-finger motifs, each one recognizing a specific three base-pair DNA sequence, have been engineered. See Hossain et al., “Artificial Zinc Finger DNA Binding Domains: Versatile Tools for Genome Engineering and Modulation of Gene Expression,” (2015) J Cell Biochem. 116(11): 2435-2444.

Zinc finger nucleases (ZFNs) are a class of engineered DNA-binding proteins that comprise a zinc finger DNA-binding domain fused to a DNA-cleavage domain. As the sequence recognized by the DNA-binding domain can be altered, ZFNs facilitate targeted editing of DNA at user-specified locations. ZFNs can be used for targeted genome editing by creating double-strand breaks in DNA having desired sequences, similar to CRISPR/Cas9 and TALEN. See, e.g., Carroll, Genome Engineering With Zinc-Finger Nucleases, Genetics. 2011 August; 188(4): 773-782.

### Cell Targeting

DNA mutations are associated with many types of disease, including cancer. However, oncogenic driver mutations, which play important roles in carcinogenesis and cancer progression, remain largely untargetable. Here, the inventors provide a programable cell targeting system that comprises a sequence specific nucleic acid targeting domain. The system can be tuned to bind desired sequences, including without limitation cancer-related mutations in extracellular DNA on the cell surface. The extracellular DNA can be derived from the cell or from surrounding cells in the local microenvironment. Such targeting can be used in diagnostic, therapeutic and other applications. In some embodiments, the system is used to deliver a payload to a cell that carries the mutation.

Current cellular targeting mechanisms, such as monoclonal antibody therapy or cellular therapies directed by antibody fragments, have revolutionized disease therapy in certain cases but suffer from limitations. As a threshold matter, these targeting approaches require that the target of the therapy is druggable (i.e., available to be targeted), such as displayed on the cell surface. Specific disease targets such as mutant TP53 protein may be prevalent in cancer cells but not healthy cells, but are nevertheless unavailable for use in targeted therapy. Moreover, diseases may be characterized by loss of certain biomarkers, e.g., due to truncations or loss of expression. It will be appreciated that absent biomarkers are not available as druggable targets. Even when druggable, the target biomarkers may be preferentially expressed on diseased cells, but are also present on non-target cells, and thus targeting therapies may have toxicities that can even result in death. For example, the canonical cancer marker HER2 is expressed in cardiac tissue and anti-HER2 monoclonal antibody therapy can lead to cardiomyopathy.

The cell targeting constructs provided herein overcome such limitations by expressly targeting nucleic acids associated with diseased cells, thereby fulfilling a long-felt unmet need. Although such constructs can target any desirable nucleic sequence, it is advantageous that the constructs can be targeted to mutant nucleic acid sequences that are only present in diseased cells. Moreover, the target nucleic acid sequences need not be expressed into proteins that make their way to the cell surface.

### SUMMARY OF THE DISCLOSURE

The present disclosure provides cell targeting constructs that target cells of interest through the use of binding agents to cell surface nucleic acids, including without limitation extracellular DNA (exDNA). The cell targeting constructs can be engineered to deliver desired payload, including without limitation therapeutic agents and/or detectable labels, to the target cells.

Provided herein is a cell targeting construct comprising a binding agent to a target nucleic acid on the surface of a target cell, wherein the binding agent comprises a protein, and wherein the binding agent is attached covalently or non-covalently to at least one payload. In some embodiments, the binding agent comprises at least one nucleic acid recognition domain which is specific to the target nucleic acid. In some embodiments, the at least one nucleic acid recognition domain comprises at least one zinc finger unit, a CRISPR-associated protein, an antibody binding domain, transcription activator-like effector nucleases (TALENs), or any useful combination thereof. In some embodiments, the at least one zinc finger unit consists of at least 1, 2, 3, 4, 5, 6, 7, 8, 9, or 10 zinc finger units. In some embodiments, the at least one zinc finger unit consists of no more than 20, 15, 12, 10, 9, 8, 7, 6, 5, 4, 3, or 2 zinc finger units. In some embodiments, the at least one zinc finger unit consists of 1, 2, 3, 4, 5, 6, 7, 8, 9, 10, 11, 12, 13, 14, 15, 16, 17, 18, 19 or 20 zinc finger units. In some embodiments, the at least one zinc finger unit consists of 6 zinc finger units. In some embodiments, the binding agent is encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11, or the binding agent is encoded by a nucleic acid sequence that encodes a protein sequence that has at least 50%, 60%, 70%, 75%, 80%, 85%, 90%, 91%, 92%, 93%, 94%, 95%, 96%, 97%, 98%, 99%, or 100% identity SEQ ID NO. 4, SEQ ID NO. 6, SEQ ID NO. 10, or SEQ ID NO. 12. In some embodiments, the binding agent comprises a protein sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. In some embodiments, the at least one payload comprises a small molecule, peptide, protein, nucleic acid, toxin, therapeutic agent, drug, chemotherapeutic agent, liposome, nanoparticle, dendrimer, detectable label, or any useful combination thereof. In some embodiments, the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label. In some embodiments, the target nucleic acid comprises DNA or RNA. In some embodiments, the RNA comprises messenger RNA (mRNA) or microRNA (miRNA). In some embodiments, the target nucleic acid originated within the target cell or within the target cell microenvironment. In some embodiments, the target nucleic acid comprises genomic DNA (gDNA). In some embodiments, the target nucleic acid has a wild-type (WT) sequence or a sequence comprising one or more mutations. In some embodiments, the one or more mutations comprise at least one single nucleotide variant (whether pathogenic or not), an insertion, a deletion, a substitution, inversion, translocation, fusion, break, loss, duplication, amplification, or repeat. In some embodiments, the one or more mutation comprises one or more cancer mutation. In some embodiments, the target nucleic acid comprises a KRAS sequence. In some embodiments, the KRAS sequence comprises a Q61H, G12D and/or G13D mutation. In some embodiments, the target nucleic acid comprises at least a portion of SEQ ID NO. 1, SEQ ID NO. 2, SEQ ID NO. 7, or SEQ ID NO. 8. In some embodiments, the target nucleic acid comprises a sequence that as at least 80, 85, 90, 91, 92, 93, 94, 95, 96, 97, 98, 99, or 100% identity to SEQ ID NO. 2 or SEQ ID NO. 8. In some embodiments, the target nucleic acid comprises a foreign nucleic acid. In some embodiments, the foreign nucleic acid: i) comprises a nucleic acid sequence from viral, bacterial, fungal or other pathogenic organisms; ii) is introduced into the cell using gene therapy; and/or iii) is introduced via genetic engineering. In some embodiments, the target cell comprises a diseased cell. In some embodiments, the disease comprises a cancer, a premalignant condition, an inflammatory disease, an immune disease, an autoimmune disease or disorder, a cardiovascular disease or disorder, a neurological disease or disorder, an infectious disease or pain. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer.

Further provided herein is a nucleic acid polymer encoding some or all of the cell targeting construct provided herein (see, e.g., description above). In some embodiments, the nucleic acid polymer encodes the binding agent. In some embodiments, the nucleic acid polymer comprises a sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11. In some embodiments, the nucleic acid polymer encodes a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. Relatedly, provided herein is an expression construct comprising the nucleic acid polymer. In some embodiments, the expression construct is a viral vector. In some embodiments, the viral vector is a lenti viral vector. In some embodiments, the expression construct is a plasmid. Still further provided herein is a cell containing the nucleic acid polymer. In some embodiments, the cell containing the nucleic acid polymer comprises the expression vector. In some embodiments, the cell containing the nucleic acid polymer is used to produce the protein encoded by the nucleic acid polymer.

Provided herein is a composition comprising the cell targeting construct provided herein (see, e.g., description above) and the target cell. In some embodiments, the cell targeting construct is bound to or is internalized within the target cell. In some embodiments, the target cell comprises a diseased cell. In some embodiments, the disease associated with the cell comprises a cancer, a premalignant condition, an inflammatory disease, an immune disease, an autoimmune disease or disorder, a cardiovascular disease or disorder, a neurological disease or disorder, an infectious disease or pain. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer. In some embodiments, the target cell is a tumor cell. In some embodiments, the target cell is within a tissue, a tumor tissue, is a cultured cell, is a circulating cell, or is a circulating tumor cell.

Further provided herein is a method comprising contacting a biological specimen with the cell targeting construct provided herein (see, e.g., description above). In some embodiments, the method further comprises detecting a presence or level of the target cell in the biological specimen, wherein the cell targeting construct is bound to or is internalized within the target cell. In some embodiments, the target cell has a disease or disorder. In some embodiments, the disease or disorder comprises a cancer, a premalignant condition, an inflammatory disease, an immune disease, an autoimmune disease or disorder, a cardiovascular disease or disorder, neurological disease or disorder, infectious disease or pain. In some embodiments, the target cell comprises a neoplastic, malignant, tumor, hyperplastic, dysplastic, and/or metastatic cell. In some embodiments, the tumor is a primary tumor or a metastatic tumor. In some embodiments, the target cell is a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell. In some embodiments, the payload of the cell targeting construct comprises a detectable label and the detecting comprises detecting the detectable label. In some embodiments, the biological specimen comprises a bodily fluid, a tissue sample or a cell culture. In some embodiments, the tissue sample comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell tissue. In some embodiments, the cell culture comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the bodily fluid comprises whole blood, serum or plasma. In some embodiments, the bodily fluid comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. In some embodiments, the presence or level is used to characterize a phenotype of the biological specimen. In some embodiments, the phenotype is a disease or disorder. In some embodiments, the characterizing comprises providing, or assisting in providing, at least one of diagnostic, prognostic and theranostic information for the disease or disorder. In some embodiments, the characterizing comprises comparing the presence or level to a reference. In some embodiments, the reference comprises the presence or level determined in a sample from at least one individual without the phenotype or from at least one individual with a different phenotype. In some embodiments, the reference is a normal reference level. In some embodiments, the biological specimen is from a subject suspected of having or being predisposed to the disease or disorder. Relatedly, provided herein is a kit comprising at least one reagent for carrying out the method described above. Also relatedly, provided herein is a use of at least one reagent for carrying out the method. In some embodiments of the kit or use, the at least one reagent comprises the cell targeting construct, a detection reagent, a secondary detection reagent, a wash buffer, an elution buffer, a solid support, and any combination thereof.

Provided herein is a method of imaging at least one cell or tissue, comprising contacting the at least one cell or tissue with the cell targeting construct provided herein (see, e.g., description above), and detecting the cell targeting construct in contact with or internalized into the at least one cell or tissue. In some embodiments, the cell targeting construct is administered to a subject prior to the detecting. In some embodiments, the detecting is performed in vitro. In some embodiments, the at least one cell or tissue comprises cells displaying mutated DNA on their surface, wherein the mutated DNA is the target nucleic acid of the cell targeting construct. In some embodiments, the at least one cell or tissue is from a subject suspected of having or being predisposed to a disease or disorder. In some embodiments, the at least one cell or tissue comprises neoplastic, malignant, tumor, hyperplastic, dysplastic, and/or metastatic cells. In some embodiments, the tumor is a primary tumor or a metastatic tumor. In some embodiments, the at least one cell or tissue comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells.

Provided herein is pharmaceutical composition comprising a therapeutically effective amount of the cell targeting construct provided herein (see, e.g., description above). In some embodiments, the pharmaceutical composition comprises a pharmaceutically acceptable excipient, carrier, and/or diluent. In some embodiments, the payload of the cell targeting construct comprises a small molecule, drug, protein, nucleic acid, toxin, therapeutic agent, or chemotherapeutic agent. In some embodiments, the payload of the cell targeting construct comprises a liposome or nanoparticle. In some embodiments, the liposome or nanoparticle carries a small molecule, protein, toxin or chemotherapeutic agent. Relatedly, provided herein is method of treating or ameliorating a disease or disorder in a subject in need thereof, comprising administering the pharmaceutical composition to the subject. Further related, provided herein is method of inducing cytotoxicity in a subject, comprising administering the pharmaceutical composition to the subject. Still further related, provided herein is a method comprising detecting a nucleic acid (e.g., genomic DNA or mRNA transcript) or protein in a biological specimen from a subject, comparing a presence or level of the nucleic acid or protein to a reference, and administering the pharmaceutical composition to the subject based on the comparison. In some embodiments, the nucleic acid or protein is indicative of a disease or disorder. In some embodiments, the disease or disorder comprises a cancer. In some embodiments, the nucleic acid is genomic DNA. In some embodiments, the nucleic acid or protein comprises a mutation. In some embodiments, the nucleic acid or protein is KRAS. In some embodiments, the kRas comprises a mutation. In some embodiments, the mutation is Q61H, G12D or G13D. In some embodiments, the administering is performed if the comparison indicates that the target nucleic acid of the cell targeting construct is present. In some embodiments, the subject has or is suspected of having a disease or disorder. In some embodiments, the disease or disorder comprises a cancer, a premalignant condition, an inflammatory disease, an immune disease, an autoimmune disease or disorder, a cardiovascular disease or disorder, neurological disease or disorder, infectious disease or pain. In some embodiments, the administering comprises at least one of intradermal, intramuscular, intraperitoneal, intravenous, subcutaneous, intranasal, epidural, oral, sublingual, intracerebral, intravaginal, transdermal, rectal, by inhalation, topical administration, or any combination thereof. In some embodiments, the pharmaceutical composition is administered contemporaneously with at least one other therapeutic agent. In some embodiments, the at least one other therapeutic agent comprises a cell targeting construct engineered to target an alternate target nucleic acid sequence. In some embodiments, the administering is not performed if the comparison indicates that the target nucleic acid of the cell targeting construct is not present.

Provided herein is a protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3 or SEQ ID NO. 5. Relatedly, provided herein is a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4 or SEQ ID NO. 6. Further provided herein is a protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 9 or SEQ ID NO. 11. Relatedly, provided herein is a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 10 or SEQ ID NO. 12. In some embodiments, the protein is attached to at least one payload. In some embodiments, the at least one payload comprises a small molecule, peptide, protein, nucleic acid, toxin, chemotherapeutic agent, liposome, nanoparticle, detectable label, or any useful combination thereof. In some embodiments, the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label. Provided here is a method comprising contacting a cell with the protein described above. The contacting can be applied in various settings such as described above.

Further provided are methods of treating cancer in a patient comprising the administration of a zinc finger protein to the patient in need thereof wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target. In any of the methods disclosed, the intracellular target is a DNA mutation specific for that cancer cell. Optionally, the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are methods of targeting or identifying cancer cells comprising using a zinc finger protein wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target, and wherein the antibody optionally is attached to a biomarker for visualization. In any of the methods disclosed, the intracellular target is a DNA mutation specific for that cancer cell. Optionally the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell. In any of the methods disclosed, the cancer cells to be targeted or identified are in vivo in a patient suspected or diagnosed with cancer or alternatively, are in an in vitro sample or biopsy from a patient suspected or diagnosed with cancer.

In addition are provided methods of inhibiting the growth of a cancer cell in a patient comprising the administration of a zinc finger protein to the patient in need thereof wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target. In any of the methods disclosed, the intracellular target is a DNA mutation specific for that cancer cell. Optionally the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are methods of diagnosing cancer in a patient comprising administration of a zinc finger protein to the patient suspected of having cancer, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the biomarker is visualized or quantified to diagnose the cancer. The cancer cell can also be a precancerous cell. Optionally, the zinc finger protein that binds a cell surface exDNA specific for a cancer cell is attached to an antibody, and wherein the antibody is attached to a biomarker for visualization.

In invention further provides compositions to treat cancer in a patient in need thereof comprising a zinc finger protein wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the compositions disclosed, the zinc finger protein that binds a cell surface exDNA specific for a cancer cell is attached to an antibody that is internalized into the cancer cell and binds an intracellular target, and wherein the antibody optionally is attached to a biomarker for visualization. In any of the compositions disclosed, the intracellular target is a DNA mutation specific for that cancer cell.

Optionally, in any of the compositions, the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are compositions for diagnosing cancer in a patient suspected of having cancer comprising a zinc finger protein, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the biomarker is visualized or quantified to diagnose the cancer. The cancer cell can be a precancerous cell. The compositions can be used to diagnose the cancer in a patient suspected of having cancer in vivo or in an in vitro sample or biopsy from a patient suspected of having cancer.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

FIG. 1A-D demonstrates the presence of extracellular DNA (exDNA) presented on the surface of breast cancer cells and that DNase I digestion can remove exDNA. Human breast cancer cell lines MDA-MB-468 (FIG. 1A, FIG. 1B) and MDA-MB-231 (FIG. 1C, FIG. 1D) cells were cultured and stained with Qubit HS dsDNA fluorescent dye. exDNA appears (arrows) on the cellular membrane of the non-treated group (FIG. 1A, FIG. 1C), but is eliminated after cells were treated with DNase I (FIG. 1B, FIG. 1D).

FIG. 2 is an example of an engineered zinc finger protein (ZFP) construct provided herein. Each zinc finger unit recognizes three nucleotides and the construct has multiple zinc finger units to provide sequence specificity. In the design depicted in the figure, six zinc finger units were placed into one protein molecule and drug conjugates or other desired tags were attached.

FIG. 3 depicts the results of a gel electrophoresis of two ZFP sequences that were cloned into pET-30a(+) vector and expressed in E. coli cells (ZFP-Ras G12D-left panel; ZFP K-Ras WI-right panel).

FIG. 4 depicts dot blot results showing that ZFPs can distinguish DNA sequences with one or two point mutations. Wild type genomic DNA (gDNA) or that carrying certain mutations was spotted onto positively charged nitrocellulose membranes. The gDNA was from the cell lines MDA-MB-468, which has wild type k-Ras, MDA-MB-231, which has heterozygous G13D k-Ras, and PANC-1, which has heterozygous G12D k-Ras. We also tested control DNA with no k-Ras sequence. ZFPs designed to bind G12D k-Ras had much stronger binding affinity to gDNA of PANC-1 than gDNA of MDA-MB-468 and MDA-MB-231 (top panel). On the other hand, ZFP that targeting wild type k-Ras exhibited higher affinity to gDNA of MDA-MB-468, comparing to gDNA of MDA-MB-231 and PANC-1 (bottom panel).

FIG. 5A-C demonstrate specificity of ZFPs binding to extracellular DNA. Both copies of the kRAS gene in AsPC-1 cells are missense mutated to encode an oncogenic G12D kRAS protein. ZFP proteins were designed to selectively bind to either wild type kRAS DNA or kRAS G12D mutant DNA. ZFP proteins with a FLAG® epitope were incubated with AsPC-1 cells and binding was visualized using a fluorescently labeled anti-FLAG® antibody. In the figures, the left column (DAPI) indicates nuclear staining, the central column indicates the ZFP used (or control), and the right column (Merge) shows a combination thereof. The rows indicate whether the ZFPs were incubated with the cells in the presence (lower) or absence (upper) of DNAase I, which will digest extracellular DNA. FIG. 5A shows that ZFP G12D that had been designed to recognize DNA with aG12D mutation binds to the exDNA on the surface of AsPC-1 cells (upper central image). The fluorescent signal is the indication of the ZFP G12D. On the lower central image of FIG. 5A, faint anti-FLAG® signal from cells incubated with DNAase I is observed, which suggests a signal derived from ZFP complexes internalized within the cell prior to DNAase treatment. FIG. 5B is the same setting as FIG. 5A but with ZFP that binds to wild type kRAS. Unlike the construct used in FIG. 5A, this shows that no significant signal was detected regardless of DNase I treatment. FIG. 5C shows a control condition that uses anti-FLAG® fluorescent antibody alone and demonstrates very little if any non-specific binding.

FIG. 6 depicts data demonstrating that expression of ZFP constructs in mammalian cells interferes with transcription of the specific target genomic DNA sequence. Transcription level of KRAS were reduced by expression of ZFP targeting the KRAS gene. DNA sequence that encode ZFPs that bind to either wild type kRAS or G12D kRAS were cloned into mammalian expression vectors and transfected into cancer cells that have wild type KRAS. After culture for 3 days, cells were harvested to test the expression level of kRAS using rtPCR. The expression of ZFPs targeting wild type KRAS reduced the expression of wild type KRAS about 25%, whereas the expression of wild type KRAS was not reduced from controls in ZFP G12D transfected cells.

### DETAILED DESCRIPTION

The present disclosure is based on the hypothesis that cells can be specifically targeted through the use of binding agents to nucleic acids on the cell surface, including without limitation extracellular DNA (exDNA). Such binding agents can be, for example, proteins that recognize target nucleic acid sequences of interest. Sequence specific binding agents to specific DNA sequences include zinc finger proteins (ZFPs), anti-nucleic acid antibodies, CRISPR-associated proteins (including without limitation Cas-9) and transcription activator-like effector nucleases (TALENs). In preferred embodiments, zinc finger domains may be used to provide sequence specificity to create cell targeting constructs. Zinc finger nucleotide recognition units can be assembled together to achieve a desired nucleic acid sequence specificity. The cell targeting constructs can be engineered to deliver desired payload to the target cells, including without limitation therapeutic agents and/or detectable labels.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The present disclosure provides cell targeting constructs that target cells of interest using binding agents to cell surface nucleic acids, including without limitation extracellular DNA (exDNA). Such binding agents can be, for example, proteins that recognize target nucleic acid sequences of interest. Sequence specific binding agents to specific DNA sequences include zinc finger proteins (ZFPs), anti-nucleic acid antibodies, CRISPR-associated proteins (including without limitation Cas-9) and transcription activator-like effectors (TALEs). See, e.g., Gaj et al., ZFN, TALEN and CRISPR/Cas-based methods for genome engineering, Trends Biotechnol. 2013 July; 31(7): 397-405; Kim and Kini, Engineering and Application of Zinc Finger Proteins and TALEs for Biomedical Research, Mol Cells. 2017 Aug. 31; 40(8): 533-541. The cell targeting constructs can be engineered to deliver desired payload to the target cells, including without limitation therapeutic agents and/or detectable labels.

Provided herein is a cell targeting construct comprising a binding agent to a target nucleic acid on the surface of a target cell. In some embodiments, the binding agent comprises a protein. In some embodiments, the binding agent is attached to at least one payload. Such attachment can be covalent, non-covalent, or both. Thus, the construct can specifically target a cell of interest having the target nucleic acid on its surface, including without limitation exDNA, and may also deliver one or more payload to the cell.

In preferred embodiments, the binding agent within the cell targeting construct comprises at least one nucleic acid recognition domain that is specific to the target nucleic acid. As described herein, various proteins that recognize specific nucleic acid sequences have been identified, including zinc finger proteins, transcription factors, and CRISPR Associated Proteins (in concert with guide RNAs). In some embodiments, the at least one nucleic acid recognition domain of the binding agent comprises at least one zinc finger unit, a CRISPR-associated protein, an antibody binding domain, transcription activator-like effector nucleases (TALENs), or any useful combination thereof. In the case of nucleases, the recognition domain of the binding agent may be engineered to attenuate or eliminate the nucleolytic activity as desired.

In preferred embodiments, zinc finger domains are used to provide sequence specificity to create the cell targeting constructs provided herein. Zinc finger domains can each recognize 3-4 nucleotides in DNA, and multiple zinc finger units (ZFU) can be assembled together to achieve a desired nucleic acid sequence specificity. As a non-limiting example, a six ZFU assembly might recognize a 18 nucleotide sequence, which is enough to provide unique sequence recognition capabilities in the human genome. See Kim and Kini (2017). Researchers have identified ZFUs that recognize the 64 possible three nucleotide sequences. Such ZFUs can then be assembled as desired without requiring laborious screening steps that could be involved in identification of antibodies to particular nucleotide sequences (and may prove unsuccessful). See id; Bhakta and Segal, The generation of zinc finger proteins by modular assembly, Methods Mol Biol. 2010; 649: 3-30.

In addition to the advantages of modular assembly, zinc finger proteins are inherently cell permeable, presumedly due to the net positive charge of the ZFUs. See, e.g., Gaj et al., Targeted gene knockout by direct delivery of ZFN proteins, Nat Methods. 2012 August; 9(8): 805-807; Gaj et al., Protein delivery using Cys₂-His₂ zinc-finger domains, ACS Chem Biol. 2014 Aug. 15; 9(8): 1662-1667. Zinc finger proteins have been demonstrated to efficiently deliver payload such as proteins intracellularly and in functional form in a variety of cell types. See id; Lui et al., Improved cell-penetrating zinc-finger nuclease proteins for precision genome engineering, Mol Ther Nucleic Acids. 2015 Mar. 10; 4(3):e232; Ren et al., In Vivo Applications of Cell-Penetrating Zinc-Finger Transcription Factors, Methods Mol Biol. 2018; 1867:239-251. Cell permeability of ZFN constructs may be enhanced by the addition of facilitating signals such as positively charged peptides, nuclear localization signals (NLS), cell-penetrating peptides (CPP) or the like. See Id.

As noted, the number of ZFUs can be chosen to reach a desired level of sequence specificity. For example, a six ZFU assembly that specifically recognizes an 18-mer nucleotide sequence may provide unique targeting capabilities to the cell targeting construct. On the other hand, a three, four or five ZFU assembly may be used for certain applications wherein unique sequence recognition is not necessary or not desired, e.g., to promiscuously recognize exDNA encoding multiple proteins, such as a given protein family or protein motif. In some embodiments of the cell targeting construct provided herein, the at least one zinc finger unit consists of or comprises at least 1, 2, 3, 4, 5, 6, 7, 8, 9, 10, 12, 15 or 20 zinc finger units. In some embodiments, the at least one zinc finger unit consists of or comprises no more than 20, 15, 12, 10, 9, 8, 7, 6, 5, 4, 3, or 2 zinc finger units. In some embodiments, the at least one zinc finger unit consists of or comprises 1, 2, 3, 4, 5, 6, 7, 8, 9, 10, 11, 12, 13, 14, 15, 16, 17, 18, 19 or 20 zinc finger units. In preferred embodiments, the number of zinc finger units within the binding agent portion of the cell target construct provided herein is between 1 and 10, e.g., between 2 and 7 or between 3 and 6. In some embodiments, the at least one zinc finger unit consists of or comprises 6 zinc finger units.

As described in the Examples below, the inventors constructed exemplary cell targeting constructs that recognize wild type (WT) or mutant KRAS, specifically to the oncogenic G12D mutation. See, e.g., Example 3 herein. The inventors showed that such constructs specifically recognize the intended target DNA and that such constructs bind to cells whose genomic DNA carries the KRAS sequence recognized by the construct. See, e.g., Examples 4-6 herein. The KRAS WT cell targeting construct, which may be referred to herein as ZFP KRAS WT (equivalently ZFP K-RAS WT, ZFP WT, or the like), is encoded by the DNA sequences in SEQ ID NO. 3 (with 3× FLAG® tag) or SEQ ID NO. 5 (without FLAG® tag). The corresponding protein sequences are shown as SEQ ID NO. 4 and SEQ ID NO. 6, respectively. See Example 3. The KRAS G12D cell targeting construct, which may be referred to herein as ZFP KRAS G12D (equivalently ZFP K-RAS G12D, ZFP G12D, or the like), is encoded by the DNA sequences in SEQ ID NO. 9 (with 3× FLAG® tag) or SEQ ID NO. 11 (without FLAG® tag). The corresponding protein sequences are shown as SEQ ID NO. 10 and SEQ ID NO. 12, respectively. See Example 3.

As described in Example 3, the FLAG® tag can be added to protein constructs and used to facilitate purification, detection, labeling, and the like through the use of anti-FLAG® antibodies. In the cell targeting constructs provided herein, including without limitation ZFP KRAS WT and ZFP KRAS G12D, the FLAG® tags can be added or removed as desired. The FLAG® tags can also be replaced with alternate tags that provide similar functionality. In some cases, the tags can be engineered such that the protein is synthesized with the tag, but the tag can be removed thereafter, such as by specific cleavage events. Such alternate tags are contemplated within the scope of the cell targeting constructs provided herein.

Accordingly, in some embodiments, the binding agent of the cell targeting construct provided herein is encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11. In some embodiments, the binding agent is encoded by a nucleic acid sequence that encodes a protein sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. In some embodiments, the binding agent comprises a protein sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. As used herein, identity refers to the extent to which two nucleotide or amino acid sequences have the same residues at the same positions in an alignment, often expressed as a percentage. See BLAST Glossary, available at www.ncbi.nlm.nih.gov/books/NBK62051/. Note that the nucleotide sequences of ZFP KRAS WT and ZFP KRAS G12D as provided herein were optimized for expression in E. coli cells. Due to the degenerate nature of the genetic code, it will be appreciated that alternate nucleic acid sequences will encode the same protein sequences. Such alternate nucleic acid sequences are contemplated within the scope of the sequences provided herein.

As noted, the cell targeting constructs provided herein can be used to deliver one or more payloads to the target cell. As used herein, a payload can be any desired molecule, complex, or other entity that can be attached to the binding agent portion of the construct. The payload may be attached covalently, including without limitation direct conjugation to the binding agent, via a linker entity, or both. In some cases, the payload may be attached non-covalently. In some embodiments, e.g., in the case of multiple payloads, the one or more payload may be attached both covalently and non-covalently. In a non-limiting example, the binding agent portion of the construct may be conjugated to a biotin moiety, and the payload could be attached to a streptavidin. In this example, the biotin-streptavidin bond would provide the non-covalent attachment between the binding agent portion and the payload. The linker can be any useful linker and can be chosen to impart a desired property. In some embodiments, the linker provides a permanent attachment. In some embodiments, the linker is chosen to release the payload under certain conditions. For example, a linker that is sensitive to pH might release the payload once the cell targeting construct is internalized within the cell. As another example, a linker that is sensitive to irradiation might be used to target the area of release, such as within a tumor in a patient. Such concepts may be combined. For example, the linker may only release the payload if certain environmental conditions are met and the area is irradiated.

Any useful and desired payload can be delivered using the cell targeting constructs provided herein. Such flexibility allows the cell targeting constructs to be used in multiple applications, such as diagnostics, prognostics or theranostics. The term “theranostics” refers to therapy-related diagnostics, including without limitation using diagnostic information to predict or monitor drug response. In some embodiments, the at least one payload comprises a small molecule, peptide, protein, nucleic acid, toxin, chemotherapeutic agent, liposome, nanoparticle, dendrimer, detectable label, or any useful combination thereof. As a non-limiting example, the small molecule could be a therapeutic agent such as a drug that is specifically delivered to a cell harboring a certain mutation using the cell targeting construct, such a tumor cell. Such an application may be intended to provide a therapeutic effect. In other embodiments, the cell targeting construct may be used to detect the target cell. In such cases, detectable labels may be desired payload. In some embodiments, the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label. In still other embodiments, payload may be used for both detection for diagnostic purposes and simultaneously for therapeutic purposes. As a non-limiting example, a radioactive label could be used to detect and/or kill target cells.

Examples of therapeutic agents that may be attached as payload to the cell targeting constructs provided herein include, but are not limited to, antitumor agents, antineoplastic agents, prodrugs, lysosome destabilizing agents (e.g., chloroquine), alkylating agents, alkaloids, allosteric inhibitors, antifolics, anti-inflammatory agents, antibiotics, antibacterials, antifungals, antifibrotic agents, anti-infective agents, anti-parasitic agents, antiviral agents, antimycobacterial agents, antineoplastic agents, antiprotozoal agents, antiviral agents, drugs, bioactive peptides, steroid hormones, nucleic acids, photosensitizer substances, radio-pharmaceuticals, antiprion agents, and combinations thereof. For example, the therapeutic agent may be an antitumor agent selected from the group consisting of an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carotenoid, or a tocopherol; a cyclooxygenase inhibitor; an MMP inhibitor; a mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a Flt-3 inhibitor; an Hsp90 inhibitor; a kinesin spindle protein inhibitor; a MEK inhibitor; an antitumor antibiotic; a nitrosourea, a compound targeting/decreasing protein or lipid kinase activity, a compound targeting/decreasing protein or lipid phosphatase activity, any further anti-angiogenic compound, and combinations thereof. Specific examples of antitumor agents include, but are not limited to, azacitidine, axathioprine, bevacizumab, bleomycin, capecitabine, carboplatin, chlorabucil, cisplatin, cyclophosphamide, cytarabine, daunorubicin, docetaxel, doxifluridine, doxorubicin, epirubicin, etoposide, fenretinide, fluorouracil, gemcitabine, herceptin, idarubicin, mechlorethamine, melphalan, mercaptopurine, methotrexate, mitoxantrone, oxaliplatin, paclitaxel, tafluposide, teniposide, tioguanine, retinoic acid, valrubicin, vinblastine, vincristine, vindesine, vinorelbine, receptor tyrosine kinase inhibitors, and combinations thereof. Additional examples of antitumor and other therapeutic agents are known in the art.

The cell targeting construct provided herein can recognize any desired nucleic acid on the cell surface. As described herein, the target nucleic acid can be one that carries information about the cell of interest from which the nucleic acid is derived. In some embodiments, the target nucleic acid comprises DNA or RNA. In preferred embodiments, the DNA is genomic DNA (gDNA). The DNA could be mitochondrial DNA. The RNA can be a coding or non-coding RNA. In some embodiments, the RNA comprises mRNA, microRNA, snoRNA, snRNA, rRNA, tRNA, siRNA, hnRNA, shRNA or lncRNA. In some embodiments, the RNA comprises messenger RNA (mRNA) or microRNA (miRNA). In some embodiments, the target nucleic acid of the cell targeting construct is more than one form of nucleic acid. For example, the target nucleic acid could be gDNA and the corresponding mRNA.

As described herein, the target nucleic acid can be chosen to allow the cell targeting construct to identify one or more cell of interest. In preferred embodiments, the target nucleic acid originated within the target cell. For example, the target nucleic acid can carry one or more mutation that identifies the target cell as a mutated or diseased cell. The present disclosure further contemplates that the target nucleic acid is derived from the target cell's microenvironment. In a non-limiting example, consider that the target cell of the cell targeting construct is a cell within a tissue, such as a tumor tissue. If the target cell is necrotic or apoptotic, it may release nucleic acids into its microenvironment, in this example the tumor microenvironment. As a result, other cells within the microenvironment may also become target cells if the released nucleic acids attach thereto. The disclosure also envisions that the target cell actively releases nucleic acid into its microenvironment, e.g., in an area of inflammation. Such necrosis, apoptosis, inflammation, or other cell damage or response may be induced by the cell or its environment (e.g., due to immune response), by the cell targeting construct, or both.

As described herein, the inventors of the present disclosure discovered that particular nucleic acids derived from a cell can be used to specifically target the desired cell. In some embodiments, the target nucleic acid has a wild-type (WT) sequence. In some embodiments, the target nucleic acid has a sequence comprising one or more mutations. As used herein, unless stated otherwise a mutation can refer to any sequence other than a “normal” wild type sequence. For example, a mutation can be a single nucleotide variant sequence (whether pathogenic or not), more than one such variant, an insertion, a deletion, a substitution, inversion, translocation, fusion, break, loss, duplication, amplification, or repeat. The cell targeting construct provided herein can recognize cells whose genomic DNA differs by a single point mutation. See, e.g., Examples 4-6. The cell targeting construct can also be targeted to a sequence that occurs from a genomic alteration, such as a sequence created by a translocation, break or loss in a sequence. In the case of a fusion resulting from a post-transcriptional event, such as splicing error, the target nucleic acid can be an mRNA. As described herein, cells may export nucleic acids such as gDNA due to inflammation, disease, or cellular damage. Thus, the levels of nucleic acids may be used to target the cell of interest. In a non-limiting example, an amplification event in cancer may produce abnormally high levels of a certain sequence. The cell targeting construct provided herein may target such amplified nucleic acids.

In some embodiments, the target nucleic acid of the cell targeting constructs provided herein comprise foreign nucleic acids. In a non-limiting example, the foreign nucleic acid is derived from a virus. For example, the virus may incorporate its genomic DNA into that of a host cell. The host cell may then be targeted using DNA sequences from the virus. In another non-limiting example, the foreign nucleic acid is derived from a bacteria, fungus or other such pathogenic organism. In still another non-limiting example, foreign nucleic acid is introduced into the cell such as by genetic engineering. For example, a gene therapy construct may be used to introduce nucleic acids into a cell of interest in order to allow targeting of such cell. The cell can be an engineered cell, such as a cell based therapy, including without limitation a CAR-T cell. See, e.g., Sterner and Sterner, CAR-T cell therapy: current limitations and potential strategies, Blood Cancer Journal volume 11, Article number: 69 (2021); Bashor et al., Engineering the next generation of cell-based therapeutics, Nature Reviews Drug Discovery, volume 21, pages 655-675 (2022). The cell targeting constructs can be used to detect, visualize, and/or eradicate such cells harboring foreign or introduced nucleic acids as desired using the methods provided herein.

In some embodiments, mutations within the target nucleic acid of the cell targeting constructs provided herein are cancer mutations. As used herein, a cancer mutation can be a mutation that is an active driver of a cancer, a mutation that inactivates a tumor suppressor, or it can be a mutation that frequently occurs in cancer cells, whether or not the mutation plays a direct or indirect role in the disease state. Cancer mutations can be selected that are identified by various means such as molecular profiling of cancer cells or review of the scientific literature, including literature archives such as PubMed or in online databases such as COSMIC, the Catalogue Of Somatic Mutations In Cancer (cancer.sanger.ac.uk/cosmic).

The cell targeting construct provided herein can be targeted to a KRAS sequence. Described further herein, the Kirsten rat sarcoma (KRAS) gene encodes the KRAS proto-oncogene. KRAS is involved in normal cell growth pathways, and a single amino acid substitution in the kRas protein can activate oncogenic signaling. kRas is commonly mutated in a variety of cancer types. Was shown to distinguish cells whose genomic DNA differs by a single point mutation in Kras. See, e.g., Examples 4-6. As an illustrative example, the 38G>A missense mutation in the nucleotide sequence of KRAS leads to an amino acid substitution at position 13 in the KRAS protein, from a glycine (G) to an aspartic acid (D). This mutation is commonly referred to as KRAS G12D. In some embodiments, common cancer mutations Q61H, G12D, G13D, and/or other mutations in KRAS, are targeted by the cell targeting constructs provided herein. Mutations in KRAS that can be targeted by the constructs include without limitation 34G>T (G12C), 34G>C (G12R), 34G>A (G12S), 35G>C (G12A), 35G>A (G12D), 35G>T (G12V), 37G>T (G13C), 37G>C (G13R), 37G>A (G13S), 38G>C (G13A), 38G>A (G13D), 38G>T (G13V), 181C>A (Q61K), 182A>T (Q61L), 182A>G (Q61R), 183A>C (Q61H), 183A>T (Q61H), 351A>C (K117N), 351A>T (K117N), 436G>C (A146P), 436G>A (A146T), and 437C>T (A146V). In some embodiments, the target nucleic acid comprises at least a portion of SEQ ID NO. 1, SEQ ID NO. 2, SEQ ID NO. 7, or SEQ ID NO. 8. SEQ ID NO. 1 and SEQ ID NO. 2 are part of the coding sequence of KRAS wild type, whereas SEQ ID NO. 7 and SEQ ID NO. 8 are part of the coding sequence of KRAS G12D. See Example 3. In some embodiments, the target nucleic acid comprises a sequence that has at least 80, 85, 90, 91, 92, 93, 94, 95, 96, 97, 98, 99, or 100% identity to SEQ ID NO. 2 or SEQ ID NO. 8.

As described herein, the cell targeting construct provided herein can be used in various applications. In non-limiting examples, the construct can be used to label the target cell or kill the target cell as desired. In some embodiments, the target cell comprises a diseased cell. The diseased cell can be within a tissue, such as a solid tumor, or it may be circulating within a body. In various embodiments, the disease comprises a cancer, a premalignant condition, an inflammatory disease, an immune disease, an autoimmune disease or disorder, a cardiovascular disease or disorder, a neurological disease or disorder, an infectious disease or pain. Cancer cells display a mutator phenotype and may harbor thousands of mutations. See, e.g., Loeb, Human cancers express mutator phenotypes: origin, consequences and targeting, Nature Reviews Cancer volume 11, pages 450-457 (2011). Any cancer cell of interest can be the target cell. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer. In some embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (e.g., brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; lung cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor. In some embodiments, the cancer comprises a breast cancer. The cancer may be in an individual diagnosed with, suffering from, at risk of developing, or suspected of having cancer. The cancer may be selected from the group comprising bladder urothelial carcinoma, breast invasive carcinoma, colon adenocarcinoma, colorectal adenocarcinoma, oseophageal carcinoma, head and neck squamous cell carcinoma, kidney rental clear cell carcinoma, kidney renal papillar cell carcinoma, liver hepatocellular carcinoma, lung adenocarcinoma, lung squamous cell carcinoma, prostate adenocarcinoma, stomach and esophageal carcinoma, thyroid carcinoma, uterine corpus endometrial carcinoma, and chronic lymphocytic leukemia. In some embodiments, the cancer harbors a mutation in Kras as described herein.

Further provided herein is a nucleic acid polymer encoding some or all of the cell targeting construct such as described above. In some embodiments, the nucleic acid polymer encodes the binding agent portion of the construct. In some embodiments, the nucleic acid polymer comprises a zinc-finger protein construct as provided herein. As described in the Examples below, the inventors constructed exemplary cell targeting constructs that recognize wild type (WT) or KRAS G12D. See, e.g., Example 3 herein. The KRAS WT cell targeting construct, which may be referred to herein as ZFP KRAS WT (equivalently ZFP K-RAS WT, ZFP WT, or the like), is encoded by the DNA sequences in SEQ ID NO. 3 (with 3× FLAG® tag) or SEQ ID NO. 5 (without FLAG® tag). The corresponding protein sequences are shown as SEQ ID NO. 4 and SEQ ID NO. 6, respectively. See Example 3. The KRAS G12D cell targeting construct, which may be referred to herein as ZFP KRAS G12D (equivalently ZFP K-RAS G12D, ZFP G12D, or the like), is encoded by the DNA sequences in SEQ ID NO. 9 (with 3× FLAG® tag) or SEQ ID NO. 11 (without FLAG® tag). The corresponding protein sequences are shown as SEQ ID NO. 10 and SEQ ID NO. 12, respectively. See Example 3. In some embodiments, the nucleic acid polymer provided herein comprises a sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11. In some embodiments, the nucleic acid polymer encodes a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. Relatedly, provided herein is an expression construct comprising the nucleic acid polymer provided above. In some embodiments, the expression construct is a viral vector. Such expression vector can be a lenti viral vector. In some embodiments, the expression construct is a plasmid. Still further provided herein is a cell containing the nucleic acid polymer described above. The cell containing the nucleic acid polymer can comprise the expression vector. In some embodiments, the cell containing the nucleic acid polymer is used to produce the protein encoded by the nucleic acid polymer.

Relatedly, provided herein is a composition comprising the cell targeting construct as described herein and the target cell. In some embodiments, the cell targeting construct is bound to or internalized within the target cell. The target cell can be a target cell as described herein, including without limitation a diseased cell, such as a cell comprising a cancer, a premalignant condition, an inflammatory disease, an immune disease, an autoimmune disease or disorder, a cardiovascular disease or disorder, a neurological disease or disorder, an infectious disease or pain. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer. The cancer can also comprise other cancers such as provided herein (e.g., see above; Examples). The target cell can be within a tissue, such as a solid tumor, or it may be circulating such as within circulation within a body. In some embodiments, the target cell is a tumor cell. In some embodiments, the target cell is within a tissue, within a tumor tissue, is a cultured cell, is a circulating cell, or is a circulating tumor cell. In some embodiments, the target cell comprises a neoplastic, malignant, tumor, hyperplastic, dysplastic, and/or metastatic cell, optionally wherein the tumor is a primary tumor or a metastatic tumor.

Provided herein is a method comprising contacting a biological specimen with the cell targeting construct provided herein, such as the constructs described above. In some embodiments, the method further comprises detecting a presence or level of one or more target cell in the biological specimen, wherein the cell targeting construct is bound to or internalized within the target cell. The method can be applied in various settings as desired. For example, the contacting can be performed in vivo or in vitro depending on the desired application of the method.

In some embodiments, the target cell within the biological specimen is related to a disease or disorder. In non-limiting examples, the disease or disorder may comprise a cancer, a premalignant condition, an inflammatory disease, an immune disease, an autoimmune disease or disorder, a cardiovascular disease or disorder, neurological disease or disorder, infectious disease or pain. In some embodiments, the target cell comprises a neoplastic, malignant, tumor, hyperplastic, dysplastic, and/or metastatic cell. In the case of a tumor cell, the tumor can be a primary tumor or a metastatic tumor. The tumor can be related to any type of cancer as desired. In some embodiments, the target cell is a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell. The cancer can also comprise other cancers such as provided herein (e.g., see above; Examples).

The biological specimen contacted with the cell targeting construct provided herein can be any desired biological specimen. The biological specimen can be contacted in vivo or in vitro. In some embodiments, the biological specimen is related to a disease or disorder. In non-limiting examples, the disease or disorder may comprise a cancer, a premalignant condition, an inflammatory disease, an immune disease, an autoimmune disease or disorder, a cardiovascular disease or disorder, neurological disease or disorder, infectious disease or pain. In some embodiments, the biological specimen comprises a bodily fluid, a tissue sample or a cell culture. The tissue sample can be any desirable tissue sample. In some embodiments, the tissue sample comprises tumor tissue, including without limitation a tumor from a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell tissue. The cancer in the tissue sample can also comprise other cancers such as provided herein (e.g., see above; Examples). Similarly, the cell culture can be any desirable cell culture. In some embodiments, the cell culture comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. The cancer in the cell culture can also comprise other cancers such as provided herein (e.g., see above; Examples). Likewise, the bodily fluid can be any useful bodily fluid. In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In preferred embodiments, the bodily fluid comprises whole blood, serum or plasma. In some embodiments, the bodily fluid comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. The cancer in the bodily fluid can also comprise other cancers such as provided herein (e.g., see above; Examples).

As described above, the method comprising contacting a biological specimen with the cell targeting construct provided herein can be applied to various applications. The choice of payload can be made according to the desired application. In some embodiments, the payload comprises a detectable label and detecting the presence or level one or more target cell in the biological specimen comprises detecting the detectable label. The detectable label can be any useful label, including without limitation those described herein. The detection can be used to query the presence or level (amount) of the target cell within the biological specimen. In some embodiments, the detected presence or level is used to characterize a phenotype of the biological specimen. A phenotype can be any observable characteristic or trait related to a specimen (including without limitation that of the subject from which the specimen is derived), such as a disease or condition, a disease stage or condition stage, susceptibility to a disease or condition, prognosis of a disease stage or condition, a physiological state, or response to therapeutics.

A phenotype can be characterized by obtaining a biological specimen and analyzing one or more analytes within the specimen. For example, characterizing a phenotype for a subject or individual may include detecting a disease or condition (including pre-symptomatic early stage detecting), determining the prognosis, diagnosis, or theranosis of a disease or condition, or determining the stage or progression of a disease or condition. Characterizing a phenotype can also include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation. The products and processes described herein allow assessment of a subject on an individual basis, which can provide benefits of more efficient and economical decisions in treatment.

In some embodiments of the methods provided herein, the phenotype that is characterized is related to a disease or disorder. The biological specimen can be from a subject suspected of having or being predisposed to the disease or disorder. The characterizing can be used in providing, or assisting in providing, at least one of diagnostic, prognostic and theranostic information for the disease or disorder. In a non-limiting example of such method as provided herein, a cell targeting construct to a cancer-related target nucleic acid may be contacted with a biological specimen from a subject. A detectable label on the cell targeting construct can be used to determine the presence or level of the target cell within the biological specimen. The presence or level may then be used to provide diagnostic (e.g., disease related cells are present or absent), prognostic (e.g., aggressive disease related cells are present or absent) and/or theranostic information (e.g., disease related cells are more or less likely to respond to a given treatment; a treatment is showing efficacy or not; etc) for the subject. The characterizing can be used to determine treatment efficacy, stage of a disease or condition, or progression of a disease or condition. For example, the amount of one or more target cells can be proportional or inversely proportional to an increase in disease stage or progression. The detected amount of target cells can be used to monitor progression of a disease or condition or to monitor a subject's response to a treatment. Such information may be useful in assisting a physician or other caregiver treating the subject.

In various embodiments of the methods provided herein, the characterization comprises comparing the presence or level of the detected target cells to a reference. In some embodiments, the reference comprises the detected presence or level determined in a sample from at least one individual without the phenotype or from at least one individual with a different phenotype. The reference can be a normal reference level.

In some embodiments, characterization includes determining whether the presence or level of the target cells are altered as compared to the reference, which reference can also be referred to a standard or a control. An alteration can include any measurable difference between the detected level and the reference, including without limitation an absolute presence or absence, a quantitative level, a relative level compared to a reference, e.g., the level of target cells present, the level of control cells, and/or the level of spiked-in markers or cells, an elevated level, a decreased level, overexpression, under expression, differential expression, a mutation, and the like.

The reference value can be from the same subject from whom a specimen is assessed, or the reference can be from a representative population of specimens (e.g., specimens from “normal” subjects without disease or not exhibiting a symptom of disease). Reference values may be set according to data pooled from groups of specimens corresponding to a particular cohort, including but not limited to age (e.g., newborns, infants, adolescents, young, middle-aged adults, seniors and adults of varied ages), racial/ethnic groups, normal versus diseased subjects, smoker v. non-smoker, subject receiving therapy versus untreated subject, different time points of treatment for a particular individual or group of subjects similarly diagnosed or treated or combinations thereof. By determining levels at different timepoints of treatment for a particular individual, the individual's response to the treatment or progression of a disease or condition for which the individual is being treated for, can be monitored.

A reference value may be based on specimens assessed from the same subject so as to provide individualized tracking. In some embodiments, frequent testing of samples from a subject provides better comparisons to the reference values previously established for that subject. Such time course measurements are used to allow a physician to more accurately assess the subject's disease stage or progression and therefore inform a better decision for treatment. In some cases, an individualized reference threshold is defined for the subject, e.g., a threshold at which a diagnosis is made. Temporal intrasubject variation allows each individual to serve as their own longitudinal control for optimum analysis of disease or physiological state. As a non-limiting example, consider that the level of target cells related to a disease is measured in a subject's blood over time. A spike in the level of target cells in the subject's blood can indicate progression of the disease.

Relatedly, provided herein is a kit comprising at least one reagent for carrying out the methods provided herein, such as those described above. Also provided herein is use of at least one reagent for carrying out the methods. Any useful reagent can be a component of the kit or use. In some embodiments, the at least one reagent comprises the cell targeting construct, a detection reagent, a secondary detection reagent, a wash buffer, an elution buffer, a solid support, and any combination thereof.

Further provided herein is a method of imaging at least one cell or tissue, comprising contacting the at least one cell or tissue with the cell targeting construct as provided herein, e.g., as described above, and detecting the cell targeting construct in contact with or internalized into the at least one cell or tissue. See, e.g., Examples 10-11 herein. In some embodiments, the cell targeting construct is administered to a subject prior to the detecting. In some embodiments, the detecting is performed in vitro. As desired, these methods can be combined. For example, the cell targeting construct can be administered to a subject, and then a sample can be taken from the subject for subsequent analysis in vitro. Although the target nucleic acid of the constructs can have any desired sequence, in preferred embodiments, the at least one cell or tissue comprises cells displaying mutated DNA on the surface, wherein the mutated DNA is the target nucleic acid of the cell targeting construct. In some embodiments, the at least one cell or tissue is from a subject suspected of having or being predisposed to a disease or disorder. In non-limiting examples, the disease or disorder may comprise a cancer, a premalignant condition, an inflammatory disease, an immune disease, an autoimmune disease or disorder, a cardiovascular disease or disorder, neurological disease or disorder, infectious disease or pain. In some embodiments, the at least one cell or tissue comprises neoplastic, malignant, tumor, hyperplastic, dysplastic, and/or metastatic cells. In the case of tumor cells, the tumor can be a primary tumor or a metastatic tumor. The tumor can be related to any type of cancer as desired. In some embodiments, the target cells or tissue comprise a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell. The cancer can also comprise other cancers such as provided herein (e.g., see above; Examples).

Still further provided herein is a pharmaceutical composition comprising a therapeutically effective amount of the cell targeting construct provided herein. The pharmaceutical composition may comprise at least one of a pharmaceutically acceptable excipient, carrier, and/or diluent.

It is contemplated that other agents may be used in combination with the pharmaceutical composition to improve the therapeutic efficacy of treatment. These additional agents include chemotherapeutic agents such as small molecule drugs or other biological agents. As desired, such additional agents may target the same biomarker as the cell targeting constructs. In some embodiments, the additional agent comprises non-targeted therapies. As a non-limiting example, a cell targeting construct directed to KRAS gDNA may be administered concurrently or sequentially with other KRAS-related therapies (e.g., afatinib, dacomitinib, erlotinib, gefitinib, 31 erceptin31e, cetuximab, and/or panitumumab), and/or traditional chemotherapy, including without limitation alkylating agents, plant alkaloids, antimetabolites, anthracyclines, topoisomerase inhibitors and/or corticosteroids.

As described herein, the payload of the cell targeting construct within the pharmaceutical composition can be selected to achieve a desired activity, such as a therapeutic effect. In some embodiments, the payload comprises a small molecule, drug, protein, nucleic acid, toxin, chemotherapeutic agent, or other therapeutic agent, such as described herein. In some embodiments, the payload comprises a liposome or nanoparticle. In such cases, the liposome or nanoparticle may carry the desired therapeutic agent inside. The cell targeting construct and/or payload may be internalized into the target cell. As described, the payload may be released in the cell to provide a therapeutic effect, e.g., via cleavage of a linker between the binding portion of the construct and the payload, via proteolytic cleavage of the binding portion, or other mechanism.

Provided herein is a method of treating or ameliorating a disease or disorder in a subject in need thereof, comprising administering a pharmaceutical composition comprising a cell targeting construct to the subject. Further provided herein is a method of inducing cytotoxicity in a subject, comprising administering a pharmaceutical composition comprising a cell targeting construct to the subject. In preferred embodiments, the pharmaceutical composition is as described above. In a non-limiting example, the cell targeting construct can comprise a zinc finger protein domain that targets a desired sequence within genomic DNA and is attached to a toxic payload such as a small molecule drug. Administration of the pharmaceutical composition may result in delivery of the payload to cells comprising the chosen sequence of genomic DNA and therefore specifically kill the target cells.

As desired, molecular profiling of one or more specimens from the subject can be performed to determine whether the subject has cells comprising the target DNA of the cell targeting construct. Such profiling can make use of any useful molecular test that provides information about the nucleic acids in the subject. In a non-limiting example, nucleic acids are extracted from a tumor sample and/or a blood sample of the subject and a sequencing method such as next-generation sequencing or polymerase chain reaction is performed to query the presence and/or level of the target DNA. Similarly, an assay could be performed to detect a gene product of the target DNA, such as the corresponding mRNA or protein. Accordingly, provided herein is method comprising detecting a nucleic acid, e.g., genomic DNA or mRNA transcript, or protein in a biological sample from a subject, comparing a presence or level of the nucleic acid or protein to a reference, and administering a pharmaceutical composition provided herein to the subject based on the comparison. In some embodiments, the nucleic acid or protein is indicative of a disease or disorder, such as a cancer or other disease or disorder provided herein. In some embodiments, the nucleic acid or protein comprises a mutation. Certain desirable mutations that may be targeted include KRAS harboring Q61H, G12D, G13D, or other KRAS mutation such as described herein. As a non-limiting example, a cell targeting construct that targets KRAS G12D may provide likely benefit to a subject having a KRAS G12D+ cancer, but not benefit a subject with a cancer that has KRAS wild type genomic DNA. In some embodiments, the administering is performed if the comparison indicates that the target nucleic acid of the cell targeting construct is present. In some embodiments, the administering is not performed if the comparison indicates that the target nucleic acid of the cell targeting construct is not present. In some embodiments, the comparison can be based on comparing the presence or level of the target DNA or a gene product thereof to a desired threshold. As a non-limiting example, a subject whose detected presence or level is above the threshold may be more likely to benefit from administration of the pharmaceutical composition that a subject whose detected presence or level is below the threshold. In the latter case, the treating physician may choose an alternate treatment regimen for the subject, including without limitation a more aggressive treatment with the cell targeting construct (e.g., higher dose, more frequent administration), an alternate treatment (including without limitation a cell targeting construct engineered to target a different target DNA), or co-administration of alternate treatments such as one or more chemotherapeutic or biological agent.

The pharmaceutical compositions provided herein, such as those described above, can be engineered to as desired to treat various diseases or disorders. In some embodiments, the disease or disorder comprises a cancer, a premalignant condition, an inflammatory disease, an immune disease, an autoimmune disease or disorder, a cardiovascular disease or disorder, neurological disease or disorder, infectious disease or pain. In some embodiments, the cells targeting by the cell targeting construct within the pharmaceutical compositions comprise neoplastic, malignant, tumor, hyperplastic, dysplastic, and/or metastatic cells. In the case of tumor cells, the tumor can be a primary tumor or a metastatic tumor. The tumor can be related to any type of cancer as desired. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer. The cancer can also comprise other cancers such as provided herein (e.g., see above; Examples).

Administration of the pharmaceutical compositions provided herein can be via any desired and useful route. This includes, but is not limited to parenteral, orthotopic, intradermal, subcutaneous, intramuscular, intraperitoneal, intranasal, or intravenous injection. In some embodiments, the route of administration comprises at least one of intradermal, intramuscular, intraperitoneal, intravenous, subcutaneous, intranasal, epidural, oral, sublingual, intracerebral, intravaginal, transdermal, rectal, by inhalation, topical administration, or any useful combination thereof.

Typically, the pharmaceutical compositions provided herein are administered in a manner compatible with the dosage formulation, and in such amount as will be therapeutically effective. The quantity to be administered depends on the subject to be treated. Precise amounts of the cell targeting construct required to be administered may depend on the judgment of the treating physician or other caregiver.

The manner of application may be varied widely. Various methods for administration of pharmaceutical compositions comprising protein components are applicable. The dosage of the pharmaceutical composition will depend on the route of administration and can vary according to the size and health of the subject.

In many instances, it will be desirable to have multiple administrations of at most about or at least about 3, 4, 5, 6, 7, 8, 9, 10 or more administrations. The timing of the administrations may vary over a time course. In some embodiments, the timing of the administrations ranges from 2-day to 12-week intervals, e.g., one to two week intervals. The course of the administrations can be followed by assays to monitor the presence and/or level of the target cells in the patients. The monitoring may be performed as described herein.

The phrase “pharmaceutically acceptable” as used herein refer to molecular entities and compositions that do not produce unacceptably adverse, allergic, or other untoward reaction when administered to a subject, e.g., a human in need of treatment for a disease or disorder. As used herein, “pharmaceutically acceptable carrier” includes any and all solvents, dispersion media, coatings, antibacterial and antifungal agents, isotonic and absorption delaying agents, and the like. The use of such media and agents for formulating pharmaceutical active substances is known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or intraperitoneal routes. Such compositions can be prepared as injectables, either as liquid solutions or suspensions. Solid forms suitable for use to prepare solutions or suspensions upon the addition of a liquid prior to injection can also be prepared. The preparations can also be emulsified.

The pharmaceutical forms suitable for injectable use include sterile aqueous solutions or dispersions; formulations including sesame oil, peanut oil, or aqueous propylene glycol. The pharmaceutical forms should be stable under the conditions of manufacture and storage and must be preserved against the contaminating action of microorganisms, such as bacteria and fungi.

Sterile injectable solutions are prepared by incorporating the active ingredients (i.e. cell targeting constructs provided herein) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the pharmaceutical composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual.

Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations can be administered in a variety of dosage forms, such as the type of injectable solutions described above.

In some embodiments, the pharmaceutical composition provided herein is administered contemporaneously with at least one other therapeutic agent. As used herein, contemporaneous administration indicates that the pharmaceutical composition and alternate treatments may be part of the same treatment regimen for a patient, but the precise timing of such administrations can be optimized. For example, the cell targeting construct and alternate treatment such as a drug or biologic may be co-administered or administered sequentially. The timing of the administration of the cell targeting construct and alternate treatment can be offset, e.g., by at least 5 min, 10 min, 15 min, 20 min, 30 min, 1 h, 2 h, 3h, 4 h, 5h, 6 h, 7h, 8 h, 9h, 10 h, 11h, 12 h, 13h, 14 h, 15h, 16 h, 17h, 18 h, 19h, 20 h, 21h, 22 h, 23h, 24 h, 30h, 36h, 2 days, 3 days, 4 days, 5 days, 6 days, 7 days, 8 days, 9 days, 10 days, 11 days, 12 days, 13 days, 14 days, 15 days, 18 days, 3 weeks, 4 weeks or more. The timing can be determined by the treating physician. In some embodiments, the at least one other therapeutic agent comprises a cell targeting construct engineered to target an alternate target nucleic acid sequence.

As described herein, the inventors have engineered zinc finger proteins that can distinguish cells whose genomic DNA differs by a single missense mutation. See, e.g., Example 5. Accordingly, provided herein is a protein encoded by a nucleic acid sequence that is at least 80, 85, 90, 91, 92, 93, 94, 95, 96, 97, 98, 99, or 100% identical to SEQ ID NO. 3 or SEQ ID NO. 5. Such nucleic acid sequence can be altered and still encode the same protein owing to the degenerate nature of the genetic code. Relatedly, provided herein is a protein having a sequence that is at least 80, 85, 90, 91, 92, 93, 94, 95, 96, 97, 98, 99, or 100% identical to SEQ ID NO. 4 or SEQ ID NO. 6. Such proteins specifically target an 18 nucleotide segment of the wild type KRAS sequence in the region of codon 12. See Example 3; SEQ ID NO. 2, for further details. Also provided herein is a method comprising contacting a cell with the protein. In some embodiments, the cell encodes the target nucleic acid of the protein.

Also provided herein is a protein encoded by a nucleic acid sequence that is at least 80, 85, 90, 91, 92, 93, 94, 95, 96, 97, 98, 99, or 100% identical to SEQ ID NO. 9 or SEQ ID NO. 11. Such nucleic acid sequence can be altered and still encode the same protein owing to the degenerate nature of the genetic code. Relatedly, provided herein is a protein having a sequence that is at least 80, 85, 90, 91, 92, 93, 94, 95, 96, 97, 98, 99, or 100% identical to SEQ ID NO. 10 or SEQ ID NO. 12. Such proteins specifically target an 18 nucleotide segment of KRAS G12D. See Example 3; SEQ ID NO. 8, for further details. Also provided herein is a method comprising contacting a cell with the protein. In some embodiments, the cell encodes the target nucleic acid of the protein. In some embodiments, the proteins provided herein are attached to at least one payload. Various useful payloads are described herein. In some embodiments, the at least one payload comprises a small molecule, peptide, protein, nucleic acid, toxin, chemotherapeutic agent, liposome, nanoparticle, detectable label, or any useful combination thereof. The detectable label can include at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label.

### EXAMPLES

The following examples, along with the methods described herein are presently representative of preferred embodiments, are provided only as examples, and are not intended as limitations on the scope of the compositions and methods provided herein. Changes therein and other uses which are encompassed within the spirit of the disclosure as defined by the scope of the claims will occur to those skilled in the art.

Materials and methods:

Cancer cell lines:

Human breast cancer cell lines (MDA-MB-231, MDA-MB-468, Hs578t) and human pancreatic cell lines (PANC-1, AsPC-1) were purchased from ATCC (Manassas, VA). MDA-MB-231, MDA-MB-468, Hs578t and PANC-1 cells were cultured at 37° C. with 5% CO₂ in Dulbecco's Modified Eagle Medium (DMEM) supplemented with 10% Fetal Bovine Serum (FBS). AsPC-1 cells were cultured at 37° C. with 5% CO₂ in Roswell Park Memorial Institute 1640 Medium (RMPI1640) supplemented with 10% FBS.

Dot blot DNA binding assay:

Genomic DNA (gDNA) was extracted from cancer cells using QIAamp DNA Mini Kit (51104, Qiagen Sciences, Inc., Germantown, MD). Concentration of the gDNA was measured by Qubit dsDNA BR Assay kit (Q32853, Invitrogen™, Thermo Fisher Scientific Inc., Waltham, MA) and adjusted to the same contraction. 400 ng of each gDNA was spotted onto a nitrocellulose membrane (15356, Sigma-Aldrich, Burlington, MA) using narrow-mouth pipette tip. Membranes were allowed to dry and then blocked by soaking in 5% non-fat milk in tris-buffered saline tween (TBS-T) at room temperature (RT) for 1 hour. Blocked membranes were incubated with ZFP (10 g/ml) dissolved in milk/TBS-T for 1 hour at RT. The membranes were washed three times with TBS-T and incubated with anti-FLAG@antibody conjugated with horseradish peroxidase (HRP) (86861S, Cell Signaling Technology, Inc., Danvers, MA). The membranes were then washed three times with TBS-T (15 min×1, 5 min×2) and once with TBS (5 min). The signals were developed with enhanced chemiluminescent (ECL) horseradish peroxidase (HRP) substrate (SuperSignal West Pico PLUS Chemiluminescent Substrate, Catalog #34580, Thermo Fisher Scientific) and captured using the ChemiDoc MP Imaging System (12003154, Bio-Rad Laboratories, Hercules, CA).

Extracellular DNA fluorescent staining

Cancer cells were seeded in 24 well plates (3526, Corning, Corning, NY) at 1×10⁵ cells/well and cultured for 48 hours. Dnase I (79254, Qiagen) was added to negative control wells and incubated for 1 hour at 37° C. Cells were washed with phosphate buffered saline (PBS) then fresh media was added. Extracellular DNA was stained with fluorescent dye (Q32854, Invitrogen) and viewed using a Lionheart™ FX Automated Microscope (Agilent Technologies, Santa Clara, CA).

### Fluorescent Imaging

Cancer cells were seeded in 24 well plates (3526, Corning) at 1×10⁵ cells/well and cultured for 48 hours. Half of the wells were treated with Dnase I (79254, Qiagen) for 1 hour as a control. The cells were incubated with 500 μL DMEM with ZFP constructs at 10 μg/ml at 37° C. for 1.5 hr. Media was removed from each well and the cells were washed with PBS and fixed with prewarmed 4% paraformaldehyde in PBS at room temperature for 10 min. Permeabilization was carried by incubating with 0.2% Triton X-100 in PBS for 10 min. The cells were then blocked with 10% normal goat serum (50062Z, Thermo Fisher Scientific) at RT for 1 hour. The cells were probed with fluorescently labeled (phycoerythrin, PE) anti-FLAG®-IgG-PE antibody in 10% normal goat serum (98533S, Cell Signaling Technology), and then counter stained with DAPI (R37606, Thermo Fisher Scientific). Cells were stored in the dark at 4° C. before visualization using a Lionheart™ FX Automated Microscope (Agilent).

Targets screening by transcription inhibition evaluation:

ZFP sequences were cloned into mammalian expression vector pCMV and then transfected into cancer cells using Lipofectamine™ 3000 (L3000001, Thermo Fisher Scientific). Cells were harvested after culture at 37° C. for 48 hours. RNAs were extracted from the harvested cells using the Rneasy Mini Kit (74104, Qiagen). TaqMan real-time PCR assays (4444557, Thermo Fisher Scientific) were performed to evaluate transcription level changes of k-Ras (Catalog #4331182, Assay ID Hs00364283_g1, Thermo Fisher Scientific). Transcription levels of IP08 (Catalog #4331182, Assay ID Hs00914057_ml, Thermo Fisher Scientific) were used as internal control. ZFP target screens were based on the premise that ZFP expressed in cancer cells binds to targets sequences in genomic DNA and thereby interferes with transcription. The affinity and specificity of a ZNF to its target sequence are evaluated by the transcription level decrease of the gene with the target sequence.

Further reference is made to the following experimental examples.

### Example 1

Extracellular DNA (exDNA) is Present on the Surface of Breast Cancer Cells

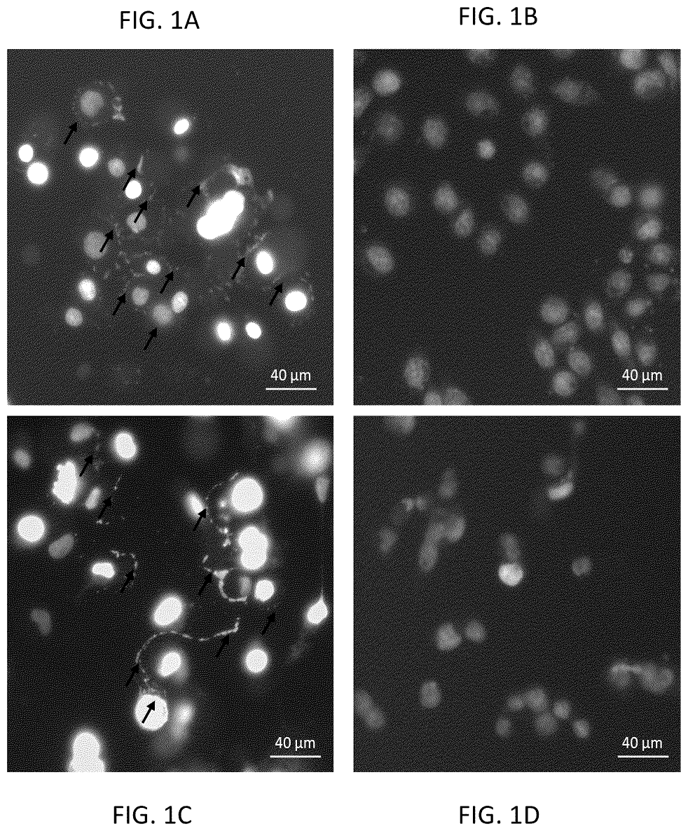

Extracellular DNA (exDNA) is found on the surface of cells in sites of inflammation and has been reported on the surface of pancreatic cancer cells. See Background herein. In this study, breast cancer cells were cultured and stained with a DNA fluorescent dye. FIGS. 1A-D demonstrate the presence of exDNA presented on the surface of breast cancer cells and that Dnase I digestion can remove exDNA. Human breast cancer cell lines MDA-MB-468 (FIG. 1A, FIG. 1B) and MDA-MB-231 (FIG. 1C, FIG. 1D) cells were cultured and stained with Qubit HS dsDNA fluorescent dye. exDNA appears (indicated in various places by arrows) on the surface of the non-treated group (FIG. 1A, FIG. 1C), but is eliminated after the cells were treated with Dnase I (FIG. 1B, FIG. 1D).

### Example 2

### Zinc Finger Protein (ZFN)

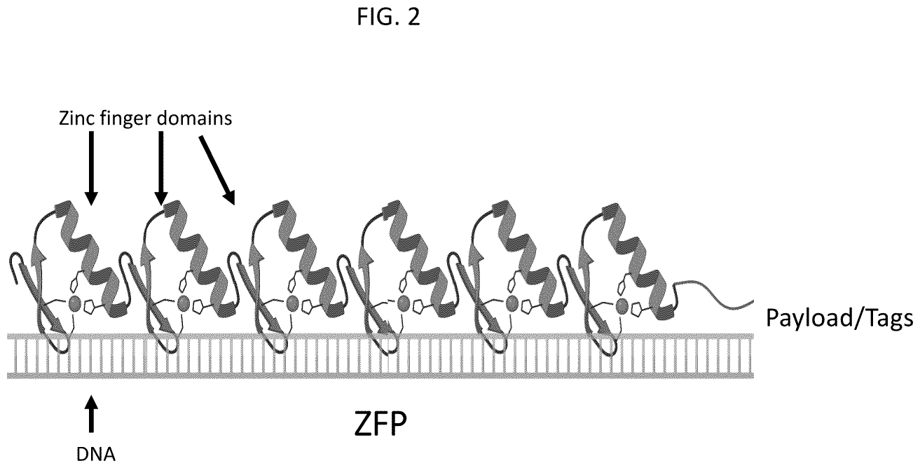

FIG. 2 is an example of a cell targeting construct provided by the present disclosure. Six zinc finger domains are depicted in the figure, wherein the domains are in contact with a double stranded DNA segment. Each zinc finger unit recognizes three nucleotides and multiple zinc finger units can be combined to provide sequence specificity. In this illustrative design, a payload/tag is attached to the zinc finger protein.

### Example 3

Construction of Zinc Finger Proteins to Target k-RAS

The KRAS (Kirsten rat sarcoma virus) gene encodes the protein K-Ras (also referred to as Kras, kRas, and the like), which is part of the RAS/MAPK signaling pathway involved in cell growth, maturation, and death. KRAS relays signals from outside the cell to the ′cell's nucleus that instruct the cell to proliferate or differentiate. The K-Ras protein is a member of the GTPase superfamily. KRAS converts guanosine-5′-triphosphate (GTP) into guanosine-5′-diphosphate (GDP). Binding of GTP to Kras activates Kras signaling, and hydrolysis of the bound GTP to GDP ′inactivates signaling.

Although wild-type KRAS may act as a tumor suppressor, oncogenic mutations in K-Ras can prevent conversion of GTP to GDP and thus constitutively activate Ras mediated signaling. See Jancik et al., “Clinical Relevance of KRAS in Human Cancers, (2010), J Biomedicine Biotech, Article ID 150960, 13 pages doi:10.1155/2010/150960. Such activating mutations in Kras are found in many types of cancers, particularly pancreatic carcinomas (>80%), colon carcinomas (40-50%), and lung carcinomas (30-50%), but also others, including biliary tract malignancies, endometrial cancer, cervical cancer, bladder cancer, liver cancer, leukemia and breast cancer. See id. Common Kras mutations that play a role in cancer are mutations in G12, e.g., G12D, G12C and G12V, G13D and Q61H. Mutations at activating hotspots in KRAS are associated with resistance to EGFR tyrosine kinase inhibitors (e.g., erlotinib, gefitinib) and monoclonal antibodies (e.g., cetuximab, panitumumab).

The epidermal growth factor receptor (EGFR) is an important player in cancer initiation and progression. KRAS plays a role as an effector molecule responsible for signal transduction from ligand-bound EGFR to the nucleus. Tumors carrying KRAS mutations are unlikely to respond to EGFR-targeted monoclonal antibodies or experience survival benefit from such treatment. EGFR directed therapy includes without limitation panitumumab, cetuximab, zalutumumab, nimotuzumab, matuzumab, gefitinib, erlotinib, and/or lapatinib.

Thus, kRas presents as a viable target for the cell targeting constructs provided herein. The inventors hypothesized that such constructs engineered to recognize wild type or mutant kRas nucleic acid sequences could be used to target cells comprising wild type or mutant KRAS exDNA. In order to test this hypothesis, two ZFPs were constructed that target an 18-nucleotide sequence from the KRAS coding region which includes the twelfth amino acid codon.

The construct “ZFP K-Ras WT” was engineered to target the section of the wild type KRAS gene sequence containing codon 12. See SEQ ID NO. 1. In this sequence as depicted below, codon 12 of KRAS (GGT) is underlined (nucleotides 34-36). The zinc finger units of ZFP K-Ras WT were engineered to recognize the particular portion of the KRAS coding region shown in SEQ ID NO. 2, wherein the nucleic acid that is missense mutated in KRAS G12D is underlined (nucleotide 7). The nucleic acid sequence encoding ZFP K-Ras WT itself is shown in SEQ ID NO. 3. This sequence is codon optimized for expression in E. coli cells, however, it will be appreciated that alternate nucleic acid sequences can encode the same amino acid sequence due to degenerate genetic coding. Finally, the predicted protein sequence of ZFP K-Ras WT is shown below in SEQ ID NO. 4. In SEQ ID NO. 3 and SEQ ID NO. 4, ZFP K-Ras WT is depicted with a 3× FLAG® epitope added to the N terminal for purification and detection purposes. The nucleic acid sequence and protein sequence of the FLAG® tag is underlined in SEQ ID NO. 3 and SEQ ID NO. 4, respectively. Such sequence provides convenience and can be removed if desired for cell targeting purposes. The nucleic acid sequence (GCGCGT) and protein sequence (AR) that differ between the ZFP K-Ras WT and ZFP K-Ras G12D constructs is underlined and italicized in SEQ ID NO. 3 and SEQ ID NO. 4, respectively. The nucleic acid sequence and protein sequence of ZFP K-Ras WT absent the FLAG® tag is shown in SEQ ID NO. 5 and SEQ ID NO. 6, respectively. Note in the sequences depicted, the 5′ end is on the left.

ZFP K-Ras WT
K-Ras WT:
(SEQ ID NO. 1)
ATGACTGAATATAAACTTGTGGTAGTTGGAGCTGGTGGCGTAGGCAAGAG
TGC
Target sequence of ZFP K-Ras WT:
(SEQ ID NO. 2)
GAGCTGGTGGCGTAGGCA
DNA sequence of ZFP K-Ras WT:
(SEQ ID NO. 3)
ATGGATTACAAGGATCACGATGGAGACTATAAGGATCACGATATAGATTA
CAAAGACGATGACGATAAGATGGCGCCGAAGAAAAAGCGTAAAGTTGGTATTCATG
GTGTTCCGGCGGCGATGGCGGAGCGTCCGTTCCAGTGCCGTATTTGCATGCGTAACT
TTAGCCAAAGCAGCGACCTGAGCCGTCACATCCGTACCCACACCGGTGAAAAACCG
TTCGCGTGCGACATTTGCGGCCGTAAGTTTGCGCGTAGCGATTATCTGCGTAAACAC
ACCAAGATCCACACCGGTAGCCAGAAGCCGTTCCAATGCCGTATTTGCATGCGCAAC
TTTAGCCGTAGCGCGGATCTGACCCGCCATATCCGTACCCACACCGGCGAGAAGCCG
TTTGCGTGCGACATTTGCGGTCGCAAATTTGCGCGTAGCGATGCGCTGGCGCGTCAC
ACCAAAATCCACACCGGCAGCCAAAAACCGTTTCAGTGTCGTATTTGCATGCGCAAT
TTTAGCGACGATTGGAACCTGAGCCAGCATATTCGCACCCATACCGGCGAGAAGCCT
TTTGCTTGTGACATTTGCGGTCGCAAGTTTGCGCGTAGCGCGAACCTGACCCGTCAC
ACCAAAATTCACCTGCGTCAAAAGGATGCGGCGCGTGGTAGCGGCGCG
Protein sequence of ZFP K-Ras WT with 3x FLAG ® epitope (underlined)
(Protein Length = 222 MW = 25665.24; predicted pI = 9.79):
(SEQ ID NO. 4)
MDYKDHDGDYKDHDIDYKDDDDKMAPKKKRKVGIHGVPAAMAERPFQCRI
CMRNFSQSSDLSRHIRTHTGEKPFACDICGRKFARSDYLRKHTKIHTGSQKPFQCRICMR
NFSRSADLTRHIRTHTGEKPFACDICGRKFARSDALARHTKIHTGSQKPFQCRICMRNFSD
DWNLSQHIRTHTGEKPFACDICGRKFARSANLTRHTKIHLRQKDAARGSGA
DNA sequence of ZFP K-Ras WT absent 3x FLAG® sequence:
(SEQ ID NO. 5)
ATGGCGCCGAAGAAAAAGCGTAAAGTTGGTATTCATGGTGTTCCGGCGGC
GATGGCGGAGCGTCCGTTCCAGTGCCGTATTTGCATGCGTAACTTTAGCCAAAGCAG
CGACCTGAGCCGTCACATCCGTACCCACACCGGTGAAAAACCGTTCGCGTGCGACAT
TTGCGGCCGTAAGTTTGCGCGTAGCGATTATCTGCGTAAACACACCAAGATCCACAC
CGGTAGCCAGAAGCCGTTCCAATGCCGTATTTGCATGCGCAACTTTAGCCGTAGCGC
GGATCTGACCCGCCATATCCGTACCCACACCGGCGAGAAGCCGTTTGCGTGCGACAT
TTGCGGTCGCAAATTTGCGCGTAGCGATGCGCTGGCGCGTCACACCAAAATCCACAC
CGGCAGCCAAAAACCGTTTCAGTGTCGTATTTGCATGCGCAATTTTAGCGACGATTG
GAACCTGAGCCAGCATATTCGCACCCATACCGGCGAGAAGCCTTTTGCTTGTGACAT
TTGCGGTCGCAAGTTTGCGCGTAGCGCGAACCTGACCCGTCACACCAAAATTCACCT
GCGTCAAAAGGATGCGGCGCGTGGTAGCGGCGCG
Protein sequence of ZFP K-Ras WT absent 3x FLAG ® epitope:
(SEQ ID NO. 6)
MMAPKKKRKVGIHGVPAAMAERPFQCRICMRNFSQSSDLSRHIRTHTGEKPF
ACDICGRKFARSDYLRKHTKIHTGSQKPFQCRICMRNFSRSADLTRHIRTHTGEKPFACDI
CGRKFARSDALARHTKIHTGSQKPFQCRICMRNFSDDWNLSQHIRTHTGEKPFACDICGR
KFARSANLTRHTKIHLRQKDAARGSGA

The construct “ZFP K-Ras G12D” was engineered to target the section of the gene sequence containing the GGT→GAT missense mutation in codon 12 leading to the substitution G→D. See SEQ ID NO. 7. In the sequence depicted below, codon 12 of KRAS G12D (GAT) is underlined (nucleotides 34-36). The zinc finger units of ZFP K-Ras G12D were engineered to recognize the particular portion of the KRAS coding region shown in SEQ ID NO. 8, wherein the nucleic acid that is missense mutated in KRAS G12D is underlined (nucleotide 7). The nucleic acid sequence encoding ZFP K-Ras G12D itself is shown in SEQ ID NO. 9. This sequence is codon optimized for expression in E. coli cells, however, it will be appreciated that alternate nucleic acid sequences can encode the same amino acid sequence due to degenerate genetic coding. Finally, the predicted protein sequence of ZFP K-Ras G12D is shown below in SEQ ID NO. 10. In SEQ ID NO. 9 and SEQ ID NO. 10, ZFP K-Ras G12D is depicted with a 3× FLAG® epitope added to the N terminal for purification and detection purposes. The nucleic acid sequence and protein sequence of the FLAG® tag is underlined in SEQ ID NO. 9 and SEQ ID NO. 10, respectively. Such sequence provides convenience and can be removed if desired for cell targeting purposes. The nucleic acid sequence (ACCCAG) and protein sequence (TQ) that differ between the ZFP K-Ras WT and ZFP K-Ras G12D constructs is underlined and italicized in SEQ ID NO. 9 and SEQ ID NO. 10, respectively. The nucleic acid sequence and protein sequence of ZFP K-Ras G12D absent the FLAG® tag is shown in SEQ ID NO. 11 and SEQ ID NO. 12, respectively.

ZFP K-Ras G12D
K-Ras G12D:
(SEQ ID NO. 7)
ATGACTGAATATAAACTTGTGGTAGTTGGAGCTGATGGCGTAGGCAAGAG
TGC
Target sequence of ZFP K-Ras G12D:
(SEQ ID NO. 8)
5'-GAGCTGATGGCGTAGGCA
DNA sequence of ZFP K-Ras G12D:
(SEQ ID NO. 9)
ATGGATTACAAGGATCACGATGGAGACTATAAGGATCACGATATAGATTA
CAAAGACGATGACGATAAGATGGCGCCGAAGAAAAAGCGTAAAGTTGGTATTCATG
GTGTTCCGGCGGCGATGGCGGAGCGTCCGTTCCAGTGCCGTATTTGCATGCGTAACT
TTAGCCAAAGCAGCGACCTGAGCCGTCACATCCGTACCCACACCGGTGAAAAACCG
TTCGCGTGCGACATTTGCGGCCGTAAGTTTGCGCGTAGCGATTATCTGCGTAAACAC
ACCAAGATCCACACCGGTAGCCAGAAGCCGTTCCAATGCCGTATTTGCATGCGCAAC
TTTAGCCGTAGCGCGGATCTGACCCGCCATATCCGTACCCACACCGGCGAGAAGCCG
TTTGCGTGCGACATTTGCGGTCGCAAATTTGCGCGTAGCGATGCGCTGACCCAGCAC
ACCAAAATCCACACCGGCAGCCAAAAACCGTTTCAGTGTCGTATTTGCATGCGCAAT
TTTAGCGACGATTGGAACCTGAGCCAACATATTCGCACCCATACCGGCGAGAAGCCT
TTTGCTTGTGACATTTGCGGTCGCAAGTTTGCGCGTAGCGCGAACCTGACCCGTCAC
ACCAAAATTCACCTGCGTCAGAAGGATGCGGCGCGTGGTAGCGGCGCG
Protein sequence of ZFP K-Ras G12D with 3x FLAG ® epitope (underlined)
(Protein Length = 222 MW = 25667.2; predicted pI = 9.73):
(SEQ ID NO. 10)
MDYKDHDGDYKDHDIDYKDDDDKMAPKKKRKVGIHGVPAAMAERPFQCRI
CMRNFSQSSDLSRHIRTHTGEKPFACDICGRKFARSDYLRKHTKIHTGSQKPFQCRICMR
NFSRSADLTRHIRTHTGEKPFACDICGRKFARSDALTQHTKIHTGSQKPFQCRICMRNFSD
DWNLSQHIRTHTGEKPFACDICGRKFARSANLTRHTKIHLRQKDAARGSGA
DNA sequence of ZFP K-Ras G12D without 3x FLAG ® tag:
(SEQ ID NO. 11)
ATGGCGCCGAAGAAAAAGCGTAAAGTTGGTATTCATGGTGTTCCGGCGGC
GATGGCGGAGCGTCCGTTCCAGTGCCGTATTTGCATGCGTAACTTTAGCCAAAGCAG
CGACCTGAGCCGTCACATCCGTACCCACACCGGTGAAAAACCGTTCGCGTGCGACAT
TTGCGGCCGTAAGTTTGCGCGTAGCGATTATCTGCGTAAACACACCAAGATCCACAC
CGGTAGCCAGAAGCCGTTCCAATGCCGTATTTGCATGCGCAACTTTAGCCGTAGCGC
GGATCTGACCCGCCATATCCGTACCCACACCGGCGAGAAGCCGTTTGCGTGCGACAT
TTGCGGTCGCAAATTTGCGCGTAGCGATGCGCTGACCCAGCACACCAAAATCCACAC
CGGCAGCCAAAAACCGTTTCAGTGTCGTATTTGCATGCGCAATTTTAGCGACGATTG
GAACCTGAGCCAACATATTCGCACCCATACCGGCGAGAAGCCTTTTGCTTGTGACAT
TTGCGGTCGCAAGTTTGCGCGTAGCGCGAACCTGACCCGTCACACCAAAATTCACCT
GCGTCAGAAGGATGCGGCGCGTGGTAGCGGCGCG
Protein sequence of ZFP K-Ras G12D without 3x FLAG ® epitope:
(SEQ ID NO. 12)
MMAPKKKRKVGIHGVPAAMAERPFQCRICMRNFSQSSDLSRHIRTHTGEKPFACDICGR
KFARSDYLRKHTKIHTGSQKPFQCRICMRNFSRSADLTRHIRTHTGEKPFACDICGRKFA
RSDALTQHTKIHTGSQKPFQCRICMRNFSDDWNLSQHIRTHTGEKPFACDICGRKFARSA
NLTRHTKIHLRQKDAARGSGA

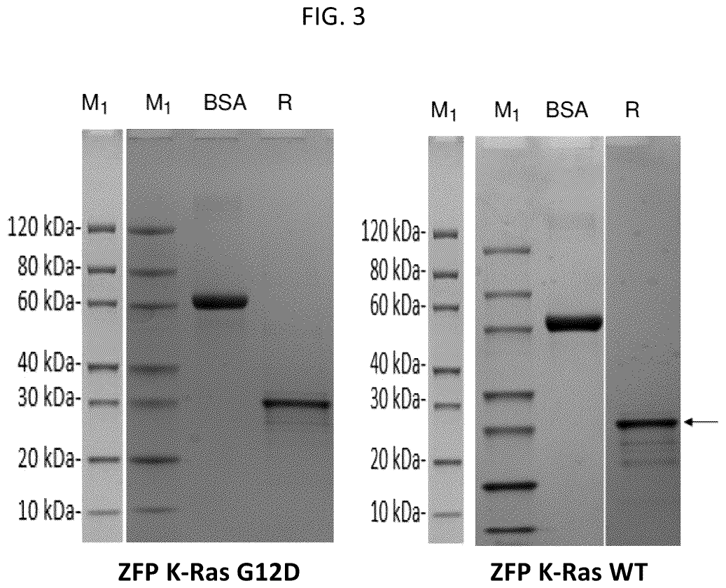

FIG. 3 depicts the results of a gel electrophoresis of the ZFP K-Ras WT and ZFP K-Ras G12D sequences cloned into pET-30a(+) vector and expressed in E coli. ZFP proteins were purified and stored in PBS with 10% glycerol, 0.5 M NaCl, and 1 mM (tris(2-carboxyethyl)phosphine) (TCEP) at −80° C.

### Example 4

Zinc Finger Proteins Targeting k-RAS Distinguish Specific DNA Sequences

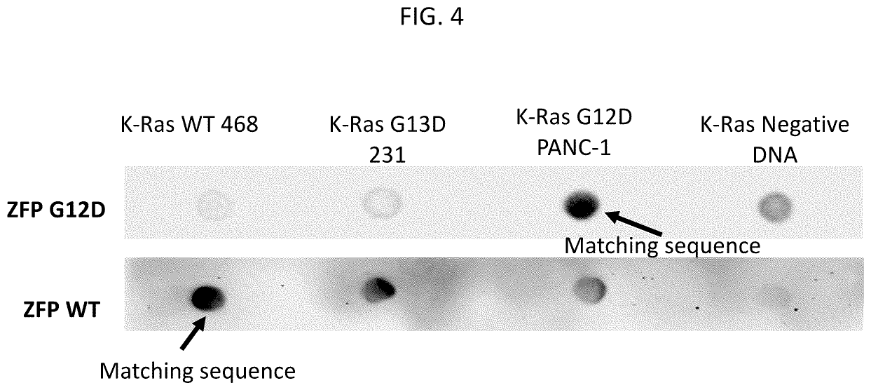

FIG. 4 depicts dot blot results showing that the ZFPs from Example 3 can distinguish DNA sequences with one or two point mutations in vitro. The specificity of the ZFPs for target nucleic acids were assayed by incubating ZFP constructs with genomic DNA containing KRAS WT, G12D, or G13D using dot blot assays. Wild type genomic DNA (gDNA) or that carrying certain mutations was spotted onto positively charged nitrocellulose membranes. The gDNA was from the cell lines MDA-MB-468, which has wild type KRAS, MDA-MB-231, which has heterozygous G13D KRAS, and PANC-1, which has heterozygous G12D KRAS. We also tested control DNA with no KRAS sequence. ZFP K-Ras G12D (upper blots) had much higher binding affinity to gDNA of PANC-1 (carrying KRAS G12D) than gDNA of MDA-MB-468 (WT KRAS) or MDA-MB-231 (KRAS G13D). On the other hand, ZFP K-Ras WT (lower blots) exhibited higher affinity to gDNA of MDA-MB-468 (WT KRAS), comparing to KRAS mutant gDNA derived from MDA-MB-231 and PANC-1.

### Example 5

Zinc Finger Proteins Specifically Bind to Cells Displaying Target exDNA

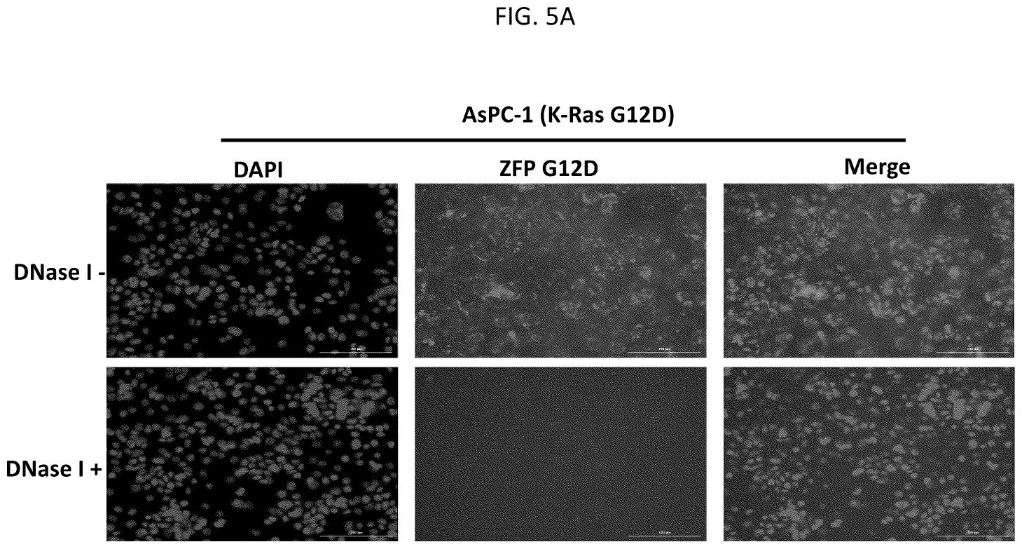

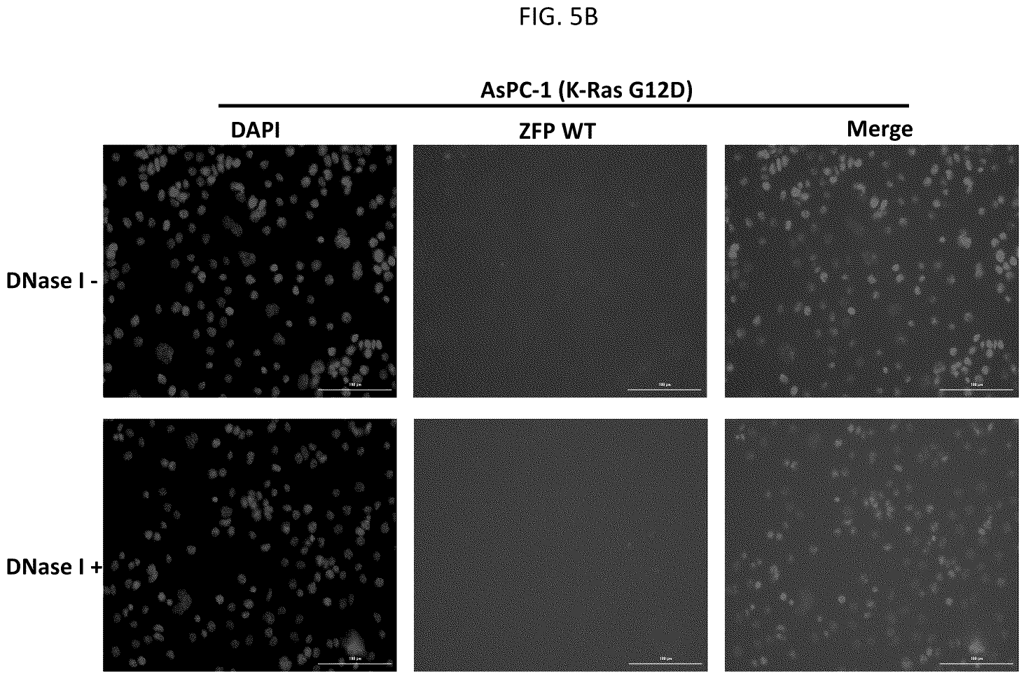

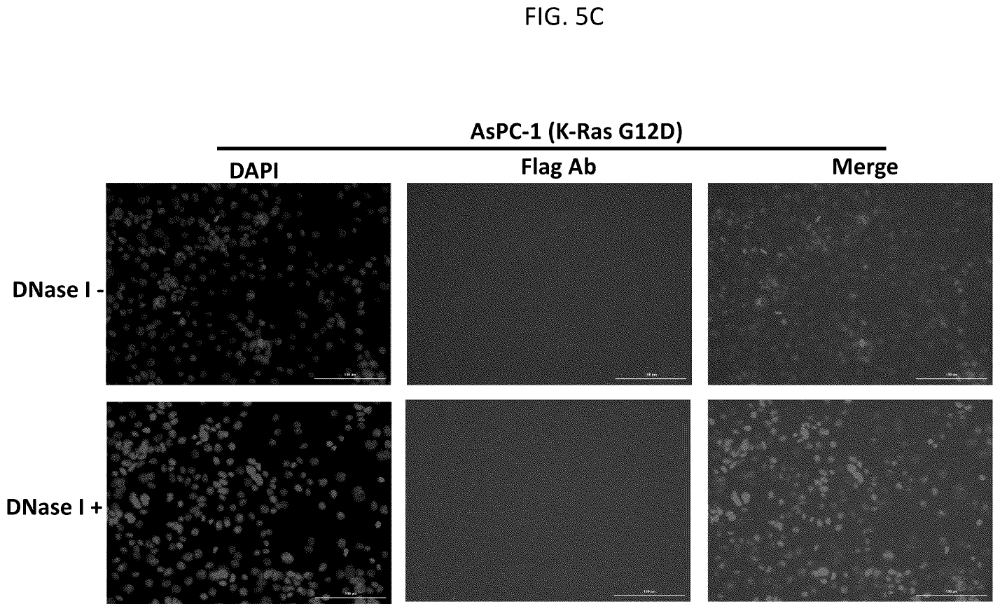

FIG. 5A-C demonstrate the specificity of ZFPs binding to extracellular DNA. These data reveal that mutations present in the genome are found in the exDNA on the cell surface and can be used to target the cells carrying such mutations. Both copies of the KRAS gene in AsPC-1 cells are missense mutated to encode an oncogenic G12D kRAS protein. AsPC-1 cells were contacted with either the construct ZFP K-Ras G12D, which was designed to selectively bind to the wild type KRAS gene sequence, or the construct ZFP K-Ras G12D, which was designed to selectively bind to the KRAS G12D gene sequence. Both constructs are described above. See, e.g., Example 3. As noted above, both constructs carry a FLAG® tag which facilitates recognition of the constructs using an anti-FLAG® antibody. The anti-FLAG® antibody used in this example is fluorescently labeled, thus allowing detection of the anti-FLAG® antibody bound ZFP constructs. To carry out these experiments, the ZFP constructs and fluorescently labeled anti-FLAG® antibody were incubated with AsPC-1 cells in culture. In the figures, the left column (DAPI) indicates nuclear staining, the central column indicates fluorescent signal from the noted ZFP (or anti-FLAG® antibody control), and the right column (Merge) shows an overlay thereof. The rows indicate whether the ZFPs were incubated with the cells in the presence (lower) or absence (upper) of DNAase I.

FIG. 5A shows that ZFP K-Ras G12D binds to the exDNA on the surface of AsPC-1 cells harboring the KRAS G12D mutation (upper central image). Although strong signal derived from the ZFP is observed on the cell surface (upper central image), faint anti-FLAG® signal from cells incubated with DNAase I is observed (lower central image), which is consistent with signal derived from ZFP complexes internalized within the cell prior to DNAase treatment. FIG. 5B is the same setting as FIG. 5A, but the ZFP construct was ZFP KRAS WT, which binds specifically to wild type KRAS nucleic acid sequence. Unlike in FIG. 5A, FIG. 5B shows little to no fluorescent signal derived from the ZFP construct with (lower central image) or without Dnase I treatments (upper central image). These data demonstrate that the ZFP construct engineered to recognize wild type KRAS does not bind to cells carrying the KRAS G12D mutation. FIG. 5C is a control condition that uses fluorescent antibody without ZFP to control for the non-specific binding. Very little to no non-specific binding was observed.

Taken together, the data in FIG. 5A-C demonstrate that the cell targeting constructs provided herein have exquisite specificity for cells carrying genomic DNA that differs by only a single missense mutation, without relying upon surface display of the mutant protein or even the expression of the protein or transcripts within the cell.

### Example 6

### In Vivo Nucleic Acid Target Binding of Cell Targeting Constructs

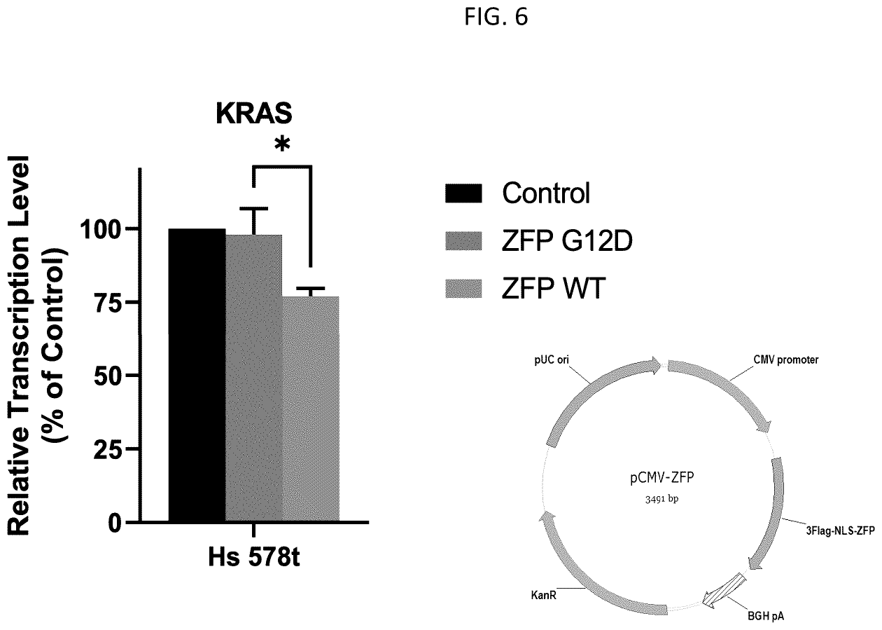

FIG. 6 demonstrates that expression of ZFP constructs in mammalian cells interferes with transcription of the specific target gene sequence. Transcription levels of KRAS were reduced by expression of ZFP targeting the K-RAS gene. In this Example, DNA sequences that encode the ZFP KRAS WT or ZFP KRAS G12D constructs (see Examples 3-5) were cloned into mammalian expression vectors and transfected into cancer cells that have wild type KRAS. The cell were cultured for 3 days then harvested to assess the expression level of kRAS using rtPCR. The expression of ZFP KRAS WT reduced the expression of wild type kRAS about 25%, which is significantly different that in the ZFP KRAS G12D transfected cells. These data further illustrate the specificity of the ZFP KRAS constructs provided herein.

### Example 7

### Diagnosing a Patient

This Example illustrates the use of the cell targeting constructs provided herein to diagnose a proliferative disease.

Cell targeting constructs are made that target KRAS G12D nucleotide sequences. The cell targeting construct consists of a binding region comprising a zinc finger domain that recognizes KRAS G12D gDNA and a payload comprising a fluorescent tag.

A tumor biopsy is taken from a breast cancer tumor. The biopsy is fixed and slides are cut. The cell targeting constructs are applied to the slide and the sample is visualized by microscope.

The visualization determines the presence, absence and/or level of KRAS G12D positive cells within the tumor sample. Visualization of the slide can also reveal presence of the KRAS G12D genomic DNA on the cell surface.

### Example 8

### Treatment Using Cell Targeting Construct

The patient in Example 6 above is determined to have KRAS G12D positive cells within the tumor sample. Such determination may also be made by sequencing nucleic acid extracted from the tumor biopsy.

The treating physician determines to treat the patient with a therapy specific for cells harboring KRAS G12D. The therapy is a pharmaceutical composition comprising a cell targeting construct consisting of a binding region comprising a zinc finger domain that recognizes KRAS G12D gDNA and a a chemotherapeutic moiety as payload.

The physician administers the pharmaceutical composition. The cell targeting construct may be co-administered with additional therapeutic agents, including without limitation small molecule drugs or biologics that target KRAS G12D protein.

### Example 9

### Monitoring Treatment Efficacy and Disease Progression

After administration of the pharmaceutical composition in Example 7, blood samples are taken from the patient over a time course, e.g., at 2-4 month increments. The cell targeting construct of Example 6 is contacted with the blood samples and used to determine the presence of circulating tumor cells carrying KRAS G12D. Flow cytometry may be used. Alternate payload may be chosen depending on the desired assay.

The presence or increased level of circulating tumor cells carrying KRAS G12D may indicate disease progression or reoccurrence.

### Example 10

### Cell Imaging

This Example describes using a cell targeting construct as an imaging agent.

The cell targeting construct is combined with imaging agents including without limitation a nanomaterial such as a magnetic nanomaterial, quantum dot, gold or radionuclide probe as desired. Sun and Zu. Aptamers and their applications in nanomedicine. Small. 2015 May; 11(20):2352-64; Dougherty C A et al., Applications of aptamers in targeted imaging: state of the art. Curr Top Med Chem. 2015; 15(12):1138-52. The nanomaterial or other imaging agent is directly conjugated to the binding agent portion of the cell targeting construct or encapsulated in a liposome or other nanoparticle. The cell targeting construct can be configured to recognize cells harboring desired nucleic acids on their surface, such as mutations in genomic DNA. The cell targeting construct is administered to a patient and imaged to visualize the location of target cells in the patient.

### Example 11

### Immunoassay or Capture

This Example illustrates immunoassays and cell capture using a cell targeting construct as provided herein. A cell targeting construct is engineered having a zinc finger binding domain specific to a desired target nucleic acid sequence. The cell targeting construct may comprise a biotin modification to facilitate specific recognition by a desired moiety attached to streptavidin.

A labeled cell targeting construct is synthesized. The biotinylated construct is contacted with fluorescently labeled streptavidin such as a streptavidin—Alexa Fluor®488 conjugate from Thermo Fisher Scientific, Catalog number: S11223. The binding agent can be directly conjugated to Alexa Fluor®488 using an appropriate linker, using labeled nanoparticles, or any other useful mechanism. This creates a fluorescently labeled cell targeting construct which is used to detect targets in various immunoassay formats. In one scenario, a biological sample known or suspected to contain a target of cell targeting is contacted with an ELISA plate. The plate is washed and contacted with the fluorescently labeled C10.36 construct. The fluorescent signal is read from the wells in the plate, thereby providing an indication of the presence or amount of target in the biological sample. In another scenario, a biological sample is directly contacted with the fluorescently labeled cell targeting construct. The contacted sample is subjected to flow cytometry to detect fluorescent particles of the size of cells, thereby providing an indication of the presence or amount of cells having surface displayed target nucleic acid in the biological sample. Alternate labels such as disclosed herein or known in the art can be used in such formats.

In another scenario, an immobilized cell targeting construct is engineered. A biotinylated construct is contacted with streptavidin conjugated beads. The beads are contacted with a biological sample known or suspected to contain a target cell of the cell targeting construct (see, e.g., Examples 3-5). The beads are precipitated (e.g., by centrifugation or magnetism) and washed. Cells that precipitate with the beads are analyzed, thereby providing an indication of the presence or amount of target in the biological sample. In another scenario, a biotinylated cell targeting construct is contacted with streptavidin agarose resin, e.g., Pierce™ Streptavidin Agarose, Thermo Fisher Scientific Catalog number: 20347 or Pierce™ High Capacity Streptavidin Agarose Thermo Fisher Scientific Catalog number: 20357. The resins are placed in a spin column or chromatography column, respectively. The cell targeting construct is contacted with the resin where it is bound by the streptavidin. A biological sample known or suspected to comprise target cells of cell targeting construct is allowed to pass through the resin. Target cells in the biological sample are retained by the aptamer within the resin and are then analyzed after elution. In either scenario, if desired, the cell targeting construct is contacted with the biological sample in solution and then the sample is contacted with the beads or resin. This step allows the cell targeting construct and target to bind freely in solution prior to aptamer immobilization.

Various modifications of the above scenarios are performed. For example, the cell targeting construct is directly conjugated to a bead or other desired surface.

As will be appreciated from the descriptions herein, a wide variety of aspects and embodiments are contemplated by the present disclosure, examples of which include, without limitation, the aspects and embodiments listed below:

Cell targeting constructs that target cells of interest through the use of binding agents to cell surface nucleic acids, including without limitation extracellular DNA (exDNA). The cell targeting constructs can be engineered to deliver desired payload, including without limitation therapeutic agents and/or detectable labels, to the target cells.

Provided herein is a cell targeting construct comprising a binding agent to a target nucleic acid on the surface of a target cell, wherein the binding agent comprises a protein, and wherein the binding agent is attached covalently or non-covalently to at least one payload. In some embodiments, the binding agent comprises at least one nucleic acid recognition domain which is specific to the target nucleic acid. In some embodiments, the at least one nucleic acid recognition domain comprises at least one zinc finger unit, a CRISPR-associated protein, an antibody binding domain, transcription activator-like effector nucleases (TALENs), or any useful combination thereof. In some embodiments, the at least one zinc finger unit consists of at least 1, 2, 3, 4, 5, 6, 7, 8, 9, or 10 zinc finger units. In some embodiments, the at least one zinc finger unit consists of no more than 20, 15, 12, 10, 9, 8, 7, 6, 5, 4, 3, or 2 zinc finger units. In some embodiments, the at least one zinc finger unit consists of 1, 2, 3, 4, 5, 6, 7, 8, 9, 10, 11, 12, 13, 14, 15, 16, 17, 18, 19 or 20 zinc finger units. In some embodiments, the at least one zinc finger unit consists of 6 zinc finger units. In some embodiments, the binding agent is encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11, or the binding agent is encoded by a nucleic acid sequence that encodes a protein sequence that has at least 50%, 60%, 70%, 75%, 80%, 85%, 90%, 91%, 92%, 93%, 94%, 95%, 96%, 97%, 98%, 99%, or 100% identity SEQ ID NO. 4, SEQ ID NO. 6, SEQ ID NO. 10, or SEQ ID NO. 12. In some embodiments, the binding agent comprises a protein sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. In some embodiments, the at least one payload comprises a small molecule, peptide, protein, nucleic acid, toxin, therapeutic agent, drug, chemotherapeutic agent, liposome, nanoparticle, dendrimer, detectable label, or any useful combination thereof. In some embodiments, the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label. In some embodiments, the target nucleic acid comprises DNA or RNA. In some embodiments, the RNA comprises messenger RNA (mRNA) or microRNA (miRNA). In some embodiments, the target nucleic acid originated within the target cell or within the target cell microenvironment. In some embodiments, the target nucleic acid comprises genomic DNA (gDNA). In some embodiments, the target nucleic acid has a wild-type (WT) sequence or a sequence comprising one or more mutations. In some embodiments, the one or more mutations comprise at least one single nucleotide variant (whether pathogenic or not), an insertion, a deletion, a substitution, inversion, translocation, fusion, break, loss, duplication, amplification, or repeat. In some embodiments, the one or more mutation comprises one or more cancer mutation. In some embodiments, the target nucleic acid comprises a KRAS sequence. In some embodiments, the KRAS sequence comprises a Q61H, G12D and/or G13D mutation. In some embodiments, the target nucleic acid comprises at least a portion of SEQ ID NO. 1, SEQ ID NO. 2, SEQ ID NO. 7, or SEQ ID NO. 8. In some embodiments, the target nucleic acid comprises a sequence that as at least 80, 85, 90, 91, 92, 93, 94, 95, 96, 97, 98, 99, or 100% identity to SEQ ID NO. 2 or SEQ ID NO. 8. In some embodiments, the target nucleic acid comprises a foreign nucleic acid. In some embodiments, the foreign nucleic acid: i) comprises a nucleic acid sequence from viral, bacterial, fungal or other pathogenic organisms; ii) is introduced into the cell using gene therapy; and/or iii) is introduced via genetic engineering. In some embodiments, the target cell comprises a diseased cell. In some embodiments, the disease comprises a cancer, a premalignant condition, an inflammatory disease, an immune disease, an autoimmune disease or disorder, a cardiovascular disease or disorder, a neurological disease or disorder, an infectious disease or pain. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer.

Further provided herein is a nucleic acid polymer encoding some or all of the cell targeting construct provided herein (see, e.g., description above). In some embodiments, the nucleic acid polymer encodes the binding agent. In some embodiments, the nucleic acid polymer comprises a sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11. In some embodiments, the nucleic acid polymer encodes a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12. Relatedly, provided herein is an expression construct comprising the nucleic acid polymer. In some embodiments, the expression construct is a viral vector. In some embodiments, the viral vector is a lenti viral vector. In some embodiments, the expression construct is a plasmid. Still further provided herein is a cell containing the nucleic acid polymer. In some embodiments, the cell containing the nucleic acid polymer comprises the expression vector. In some embodiments, the cell containing the nucleic acid polymer is used to produce the protein encoded by the nucleic acid polymer.

Provided herein is a composition comprising the cell targeting construct provided herein (see, e.g., description above) and the target cell. In some embodiments, the cell targeting construct is bound to or is internalized within the target cell. In some embodiments, the target cell comprises a diseased cell. In some embodiments, the disease associated with the cell comprises a cancer, a premalignant condition, an inflammatory disease, an immune disease, an autoimmune disease or disorder, a cardiovascular disease or disorder, a neurological disease or disorder, an infectious disease or pain. In some embodiments, the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer. In some embodiments, the target cell is a tumor cell. In some embodiments, the target cell is within a tissue, a tumor tissue, is a cultured cell, is a circulating cell, or is a circulating tumor cell.

Further provided herein is a method comprising contacting a biological specimen with the cell targeting construct provided herein (see, e.g., description above). In some embodiments, the method further comprises detecting a presence or level of the target cell in the biological specimen, wherein the cell targeting construct is bound to or is internalized within the target cell. In some embodiments, the target cell has a disease or disorder. In some embodiments, the disease or disorder comprises a cancer, a premalignant condition, an inflammatory disease, an immune disease, an autoimmune disease or disorder, a cardiovascular disease or disorder, neurological disease or disorder, infectious disease or pain. In some embodiments, the target cell comprises a neoplastic, malignant, tumor, hyperplastic, dysplastic, and/or metastatic cell. In some embodiments, the tumor is a primary tumor or a metastatic tumor. In some embodiments, the target cell is a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell. In some embodiments, the payload of the cell targeting construct comprises a detectable label and the detecting comprises detecting the detectable label. In some embodiments, the biological specimen comprises a bodily fluid, a tissue sample or a cell culture. In some embodiments, the tissue sample comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell tissue. In some embodiments, the cell culture comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the bodily fluid comprises whole blood, serum or plasma. In some embodiments, the bodily fluid comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells. In some embodiments, the presence or level is used to characterize a phenotype of the biological specimen. In some embodiments, the phenotype is a disease or disorder. In some embodiments, the characterizing comprises providing, or assisting in providing, at least one of diagnostic, prognostic and theranostic information for the disease or disorder. In some embodiments, the characterizing comprises comparing the presence or level to a reference. In some embodiments, the reference comprises the presence or level determined in a sample from at least one individual without the phenotype or from at least one individual with a different phenotype. In some embodiments, the reference is a normal reference level. In some embodiments, the biological specimen is from a subject suspected of having or being predisposed to the disease or disorder. Relatedly, provided herein is a kit comprising at least one reagent for carrying out the method described above. Also relatedly, provided herein is a use of at least one reagent for carrying out the method. In some embodiments of the kit or use, the at least one reagent comprises the cell targeting construct, a detection reagent, a secondary detection reagent, a wash buffer, an elution buffer, a solid support, and any combination thereof.

Provided herein is a method of imaging at least one cell or tissue, comprising contacting the at least one cell or tissue with the cell targeting construct provided herein (see, e.g., description above), and detecting the cell targeting construct in contact with or internalized into the at least one cell or tissue. In some embodiments, the cell targeting construct is administered to a subject prior to the detecting. In some embodiments, the detecting is performed in vitro. In some embodiments, the at least one cell or tissue comprises cells displaying mutated DNA on their surface, wherein the mutated DNA is the target nucleic acid of the cell targeting construct. In some embodiments, the at least one cell or tissue is from a subject suspected of having or being predisposed to a disease or disorder. In some embodiments, the at least one cell or tissue comprises neoplastic, malignant, tumor, hyperplastic, dysplastic, and/or metastatic cells. In some embodiments, the tumor is a primary tumor or a metastatic tumor. In some embodiments, the at least one cell or tissue comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells.

Provided herein is pharmaceutical composition comprising a therapeutically effective amount of the cell targeting construct provided herein (see, e.g., description above). In some embodiments, the pharmaceutical composition comprises a pharmaceutically acceptable excipient, carrier, and/or diluent. In some embodiments, the payload of the cell targeting construct comprises a small molecule, drug, protein, nucleic acid, toxin, therapeutic agent, or chemotherapeutic agent. In some embodiments, the payload of the cell targeting construct comprises a liposome or nanoparticle. In some embodiments, the liposome or nanoparticle carries a small molecule, protein, toxin or chemotherapeutic agent. Relatedly, provided herein is method of treating or ameliorating a disease or disorder in a subject in need thereof, comprising administering the pharmaceutical composition to the subject. Further related, provided herein is method of inducing cytotoxicity in a subject, comprising administering the pharmaceutical composition to the subject. Still further related, provided herein is a method comprising detecting a nucleic acid (e.g., genomic DNA or mRNA transcript) or protein in a biological specimen from a subject, comparing a presence or level of the nucleic acid or protein to a reference, and administering the pharmaceutical composition to the subject based on the comparison. In some embodiments, the nucleic acid or protein is indicative of a disease or disorder. In some embodiments, the disease or disorder comprises a cancer. In some embodiments, the nucleic acid is genomic DNA. In some embodiments, the nucleic acid or protein comprises a mutation. In some embodiments, the nucleic acid or protein is KRAS. In some embodiments, the kRas comprises a mutation. In some embodiments, the mutation is Q61H, G12D or G13D. In some embodiments, the administering is performed if the comparison indicates that the target nucleic acid of the cell targeting construct is present. In some embodiments, the subject has or is suspected of having a disease or disorder. In some embodiments, the disease or disorder comprises a cancer, a premalignant condition, an inflammatory disease, an immune disease, an autoimmune disease or disorder, a cardiovascular disease or disorder, neurological disease or disorder, infectious disease or pain. In some embodiments, the administering comprises at least one of intradermal, intramuscular, intraperitoneal, intravenous, subcutaneous, intranasal, epidural, oral, sublingual, intracerebral, intravaginal, transdermal, rectal, by inhalation, topical administration, or any combination thereof. In some embodiments, the pharmaceutical composition is administered contemporaneously with at least one other therapeutic agent. In some embodiments, the at least one other therapeutic agent comprises a cell targeting construct engineered to target an alternate target nucleic acid sequence. In some embodiments, the administering is not performed if the comparison indicates that the target nucleic acid of the cell targeting construct is not present.

Provided herein is a protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3 or SEQ ID NO. 5. Relatedly, provided herein is a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4 or SEQ ID NO. 6. Further provided herein is a protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 9 or SEQ ID NO. 11. Relatedly, provided herein is a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 10 or SEQ ID NO. 12. In some embodiments, the protein is attached to at least one payload. In some embodiments, the at least one payload comprises a small molecule, peptide, protein, nucleic acid, toxin, chemotherapeutic agent, liposome, nanoparticle, detectable label, or any useful combination thereof. In some embodiments, the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label. Provided here is a method comprising contacting a cell with the protein described above. The contacting can be applied in various settings such as described above.

Further provided are methods of treating cancer in a patient comprising the administration of a zinc finger protein to the patient in need thereof wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target. In any of the methods disclosed, the intracellular target is a DNA mutation specific for that cancer cell. Optionally, the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are methods of targeting or identifying cancer cells comprising using a zinc finger protein wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target, and wherein the antibody optionally is attached to a biomarker for visualization. In any of the methods disclosed, the intracellular target is a DNA mutation specific for that cancer cell. Optionally the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell. In any of the methods disclosed, the cancer cells to be targeted or identified are in vivo in a patient suspected or diagnosed with cancer or alternatively, are in an in vitro sample or biopsy from a patient suspected or diagnosed with cancer.

In addition are provided methods of inhibiting the growth of a cancer cell in a patient comprising the administration of a zinc finger protein to the patient in need thereof wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the methods disclosed, the zinc finger protein that binds a cell surface exDNA specific for a cancer cell is attached to an antibody that is internalized into the cancer cell and binds an intracellular target. In any of the methods disclosed, the intracellular target is a DNA mutation specific for that cancer cell. Optionally the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are methods of diagnosing cancer in a patient comprising administration of a zinc finger protein to the patient suspected of having cancer, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the biomarker is visualized or quantified to diagnose the cancer. The cancer cell can also be a precancerous cell. Optionally, the zinc finger protein that binds a cell surface exDNA specific for a cancer cell is attached to an antibody, and wherein the antibody is attached to a biomarker for visualization.

In invention further provides compositions to treat cancer in a patient in need thereof comprising a zinc finger protein wherein i) the zinc finger protein binds to a cell surface extracellular DNA (exDNA) that is specific for a cancer cell and the zinc finger protein forms a zinc finger protein/exDNA complex with the exDNA; ii) the zinc finger protein/exDNA complex is internalized into the cancer cell; and, iii) the zinc finger protein/exDNA complex binds to an intracellular target. In any of the compositions disclosed, the zinc finger protein that binds a cell surface exDNA specific for a cancer cell is attached to an antibody that is internalized into the cancer cell and binds an intracellular target, and wherein the antibody optionally is attached to a biomarker for visualization. In any of the compositions disclosed, the intracellular target is a DNA mutation specific for that cancer cell.

Optionally, in any of the compositions, the zinc finger protein that binds a cell surface exDNA specific for a cancer cell is attached to a CRISPR sequence forming a zinc finger protein/CRISPR/exDNA complex to target specific intracellular DNA sequences, wherein the zinc finger protein/CRISPR/exDNA complex is internalized into the cancer cell. Alternatively, the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

Also provided are compositions for diagnosing cancer in a patient suspected of having cancer comprising a zinc finger protein, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the biomarker is visualized or quantified to diagnose the cancer. The cancer cell can be a precancerous cell. The compositions can be used to diagnose the cancer in a patient suspected of having cancer in vivo or in an in vitro sample or biopsy from a patient suspected of having cancer.

While embodiments of the present disclosure have been described herein, it is to be understood by those skilled in the art that such embodiments are provided by way of example only. Numerous variations, changes, and substitutions will now occur to those skilled in the art without departing from the compositions and methods provided herein. It should be understood that various alternatives to the embodiments provided herein may be employed. It is intended that the following claims define the scope thereof and that methods and structures within the scope of these claims and their equivalents be covered thereby.

1. A cell targeting construct comprising a binding agent to a target nucleic acid on the surface of a target cell, wherein the binding agent comprises a protein, and wherein the binding agent is attached covalently or non-covalently to at least one payload.

2. The cell targeting construct of claim 1, wherein the binding agent comprises at least one nucleic acid recognition domain which is specific to the target nucleic acid, wherein the at least one nucleic acid recognition domain comprises at least one zinc finger unit, a CRISPR-associated protein, an antibody binding domain, transcription activator-like effector nucleases (TALENs), or any useful combination thereof.

3. The cell targeting construct of claim 2, wherein the at least one zinc finger unit consists of at least 1, 2, 3, 4, 5, 6, 7, 8, 9, or 10 zinc finger units.

4. The cell targeting construct of claim 2 or 3, wherein the at least one zinc finger unit consists of no more than 20, 15, 12, 10, 9, 8, 7, 6, 5, 4, 3, or 2 zinc finger units.

5. The cell targeting construct of any one of claims 2-4, wherein the at least one zinc finger unit consists of 1, 2, 3, 4, 5, 6, 7, 8, 9, 10, 11, 12, 13, 14, 15, 16, 17, 18, 19 or 20 zinc finger units.

6. The cell targeting construct of claim 2, wherein the at least one zinc finger unit consists of 6 zinc finger units.

7. The cell targeting construct of any preceding claim, wherein the binding agent is encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11, or the binding agent is encoded by a nucleic acid sequence that encodes a protein sequence that has at least 50%, 60%, 70%, 75%, 80%, 85%, 90%, 91%, 92%, 93%, 94%, 95%, 96%, 97%, 98%, 99%, or 100% identity SEQ ID NO. 4, SEQ ID NO. 6, SEQ ID NO. 10, or SEQ ID NO. 12.

8. The cell targeting construct of any preceding claim, wherein the binding agent comprises a protein sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12.

9. The cell targeting construct of any preceding claim, wherein the at least one payload comprises a small molecule, peptide, protein, nucleic acid, toxin, therapeutic agent, drug, chemotherapeutic agent, liposome, nanoparticle, dendrimer, detectable label, or any useful combination thereof.

10. The cell targeting construct of claim 9, wherein the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label.

11. The cell targeting construct of any preceding claim, wherein the target nucleic acid comprises DNA or RNA, optionally wherein the RNA comprises messenger RNA (mRNA) or microRNA (miRNA).

12. The cell targeting construct of any preceding claim, wherein the target nucleic acid originated within the target cell or within the target cell microenvironment.

13. The cell targeting construct of any preceding claim, wherein the target nucleic acid comprises genomic DNA (gDNA).

14. The cell targeting construct of any preceding claim, wherein the target nucleic acid has a wild-type (WT) sequence or a sequence comprising one or more mutations, optionally wherein the one or more mutations comprise at least one single nucleotide variant (whether pathogenic or not), an insertion, a deletion, a substitution, inversion, translocation, fusion, break, loss, duplication, amplification, or repeat; and optionally wherein the one or more mutation comprises one or more cancer mutation.

15. The cell targeting construct of any preceding claim, wherein the target nucleic acid comprises a KRAS sequence, optionally wherein the KRAS sequence comprises a Q61H, G12D and/or G13D mutation.

16. The cell targeting construct of any preceding claim, wherein the target nucleic acid comprises at least a portion of SEQ ID NO. 1, SEQ ID NO. 2, SEQ ID NO. 7, or SEQ ID NO. 8.

17. The cell targeting construct of any preceding claim, wherein the target nucleic acid comprises a sequence that as at least 80, 85, 90, 91, 92, 93, 94, 95, 96, 97, 98, 99, or 100% identity to SEQ ID NO. 2 or SEQ ID NO. 8.

18. The cell targeting construct of any one of claims 1-14, wherein the target nucleic acid comprises a foreign nucleic acid, optionally wherein the foreign nucleic acid: i) comprises a nucleic acid sequence from viral, bacterial, fungal or other pathogenic organisms; ii) is introduced into the cell using gene therapy; and/or iii) is introduced via genetic engineering.

19. The cell targeting construct of any preceding claim, wherein the target cell comprises a diseased cell, optionally wherein the disease comprises a cancer, a premalignant condition, an inflammatory disease, an immune disease, an autoimmune disease or disorder, a cardiovascular disease or disorder, a neurological disease or disorder, an infectious disease or pain.

20. The cell targeting construct of claim 19, wherein the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer.

21. A nucleic acid polymer encoding some or all of the cell targeting construct of any preceding claim, optionally wherein the nucleic acid polymer encodes the binding agent.

22. The nucleic acid polymer of claim 21, wherein the nucleic acid polymer comprises a sequence that has at least 50%, 60%, 70%, 75%, 80%, 85%, 90%, 91%, 92%, 93%, 94%, 95%, 96%, 97%, 98%, 99%, or 100% identity to SEQ ID NO. 3, SEQ ID NO. 5, SEQ ID NO. 9, or SEQ ID NO. 11.

23. The nucleic acid polymer of claim 21, wherein the nucleic acid polymer encodes a protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4, SEQ ID NO. 6, SEQ ID NO. 10, or SEQ ID NO. 12.

24. A cell containing the nucleic acid polymer any one of claims 21-23.

25. A composition comprising the cell targeting construct of any one of claims 1-20 and the target cell, optionally wherein the cell targeting construct is bound to or internalized within the target cell.

26. The composition of claim 25, wherein the target cell comprises a diseased cell, optionally wherein the disease comprises a cancer, a premalignant condition, an inflammatory disease, an immune disease, an autoimmune disease or disorder, a cardiovascular disease or disorder, a neurological disease or disorder, an infectious disease or pain.

27. The composition of claim 26, wherein the cancer comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer.

28. The composition of any one of claims 25-27, wherein the target cell is a tumor cell.

29. The composition of any one of claims 25-28, wherein the target cell is within a tissue, a tumor tissue, is a cultured cell, is a circulating cell, or is a circulating tumor cell.

30. A method comprising contacting a biological specimen with the cell targeting construct of any one of claims 1-20.

31. The method of claim 30, further comprising detecting a presence or level of the target cell in the biological specimen, wherein the cell targeting construct is bound to or internalized within the target cell.

32. The method of claim 30 or 31, wherein the target cell has a disease or disorder, optionally wherein the disease or disorder comprises a cancer, a premalignant condition, an inflammatory disease, an immune disease, an autoimmune disease or disorder, a cardiovascular disease or disorder, neurological disease or disorder, infectious disease or pain.

33. The method of any one of claims 30-32, wherein the target cell comprises a neoplastic, malignant, tumor, hyperplastic, dysplastic, and/or metastatic cell, optionally wherein the tumor is a primary tumor or a metastatic tumor.

34. The method of any one of claims 30-33, wherein the target cell is a bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell.

35. The method of any one of claims 30-34, wherein the payload comprises a detectable label and the detecting comprises detecting the detectable label.

36. The method of any one of claims 30-35, wherein the biological specimen comprises a bodily fluid, a tissue sample or a cell culture.

37. The method of claim 36, wherein the tissue sample comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cell tissue.

38. The method of claim 36, wherein the cell culture comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells.

39. The method of claim 36, wherein the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, Cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood.

40. The method of claim 36, wherein the bodily fluid comprises whole blood, serum or plasma.

41. The method of claim 36, 39 or 40, wherein the bodily fluid comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells.

42. The method of any one of claims 31-41, wherein the presence or level is used to characterize a phenotype of the biological specimen.

43. The method of claim 42, wherein the phenotype is a disease or disorder.

44. The method of claim 43, wherein the characterizing comprises providing, or assisting in providing, at least one of diagnostic, prognostic and theranostic information for the disease or disorder.

45. The method of any one of claims 42-44, wherein the characterizing comprises comparing the presence or level to a reference.

46. The method of claim 45, wherein the reference comprises the presence or level determined in a sample from at least one individual without the phenotype or from at least one individual with a different phenotype, optionally wherein the reference is a normal reference level.

47. The method of any one of claims 43-46, wherein the biological specimen is from a subject suspected of having or being predisposed to the disease or disorder.

48. A kit comprising at least one reagent for carrying out the method of any of claims 30-47.

49. Use of at least one reagent for carrying out the method of any of claims 30-47.

50. The kit of claim 48 or use of claim 49, wherein the at least one reagent comprises the cell targeting construct, a detection reagent, a secondary detection reagent, a wash buffer, an elution buffer, a solid support, and any combination thereof.

51. A method of imaging at least one cell or tissue, comprising contacting the at least one cell or tissue with the cell targeting construct of any one of claims 1-20, and detecting the cell targeting construct in contact with or internalized into the at least one cell or tissue.

52. The method of claim 51, wherein the cell targeting construct is administered to a subject prior to the detecting and/or wherein the detecting is performed in vitro.

53. The method of any one of claims 51-52, wherein the at least one cell or tissue comprises cells displaying mutated DNA on their surface, wherein the mutated DNA is the target nucleic acid of the cell targeting construct.

54. The method of any one of claims 51-53, wherein the at least one cell or tissue is from a subject suspected of having or being predisposed to a disease or disorder.

55. The method of any one of claims 51-54, wherein the at least one cell or tissue comprises neoplastic, malignant, tumor, hyperplastic, dysplastic, and/or metastatic cells, optionally wherein the tumor is a primary tumor or a metastatic tumor.

56. The method of any one of claims 51-54, wherein the at least one cell or tissue comprises bladder cancer, breast cancer, colon cancer, rectal cancer, endometrial cancer, ovarian cancer, kidney cancer, leukemia, liver cancer, lung cancer, melanoma, lymphoma, pancreatic cancer, prostate cancer or thyroid cancer cells.

57. A pharmaceutical composition comprising a therapeutically effective amount of the cell targeting construct according to any one of claims 1-20, optionally wherein the pharmaceutical composition comprises a pharmaceutically acceptable excipient, carrier, and/or diluent.

58. The pharmaceutical composition of claim 57, wherein the payload of the cell targeting construct comprises a small molecule, drug, protein, nucleic acid, toxin, therapeutic agent, or chemotherapeutic agent.

59. The pharmaceutical composition of claim 57, wherein the payload of the cell targeting construct comprises a liposome or nanoparticle, optionally wherein the liposome or nanoparticle carries a small molecule, protein, toxin or chemotherapeutic agent.

60. A method of treating or ameliorating a disease or disorder in a subject in need thereof, comprising administering the pharmaceutical composition of any one of claims 57-59 to the subject.

61. A method of inducing cytotoxicity in a subject, comprising administering the pharmaceutical composition of any one of claims 57-59 to the subject.

62. A method comprising detecting a nucleic acid, transcript or protein in a biological specimen from a subject, comparing a presence or level of the nucleic acid, transcript or protein to a reference, and administering the pharmaceutical composition of any one of claims 57-59 to the subject based on the comparison.

63. The method of claim 62, wherein the nucleic acid, transcript or protein is indicative of a disease or disorder, optionally wherein the disease or disorder comprises a cancer, and optionally wherein the nucleic acid is genomic DNA.

64. The method of claim 62 or 63, wherein the nucleic acid, transcript or protein comprises a mutation.

65. The method of any one of claims 62-64, wherein the nucleic acid, transcript or protein is kRas, wherein optionally the kRas comprises a mutation, wherein optionally the mutation is Q61H, G12D or G13D.

66. The method of any one of claims 62-65, wherein the administering is performed if the comparison indicates that the target nucleic acid of the cell targeting construct is present.

67. The method of any one of claims 60-66, wherein the subject has or is suspected of having a disease or disorder, wherein optionally the disease or disorder comprises a cancer, a premalignant condition, an inflammatory disease, an immune disease, an autoimmune disease or disorder, a cardiovascular disease or disorder, neurological disease or disorder, infectious disease or pain.

68. The method of any one of claims 60-67, wherein the administering comprises at least one of intradermal, intramuscular, intraperitoneal, intravenous, subcutaneous, intranasal, epidural, oral, sublingual, intracerebral, intravaginal, transdermal, rectal, by inhalation, topical administration, or any combination thereof.

69. The method of any one of claims 60-68, wherein the pharmaceutical composition is administered contemporaneously with at least one other therapeutic agent, optionally wherein the at least one other therapeutic agent comprises a cell targeting construct engineered to target an alternate target nucleic acid sequence.

70. The method of any one of claims 62-69, wherein the administering is not performed if the comparison indicates that the target nucleic acid of the cell targeting construct is not present.

71. A protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 3 or SEQ ID NO. 5.

72. A protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 4 or SEQ ID NO. 6.

73. A protein encoded by a nucleic acid sequence that has at least 50%, 60%, 70%, 75%, 80%, 85%, 90%, 91%, 92%, 93%, 94%, 95%, 96%, 97%, 98%, 99%, or 100% identity to SEQ ID NO. 9 or SEQ ID NO. 11.

74. A protein having a sequence that has at least 50%, 60%, 70%, 75%, 80%, 85%, 90%, 91%, 92%, 93%, 94%, 95%, 96%, 97%, 98%, 99%, or 100% identity to SEQ ID NO. 10 or SEQ ID NO. 12.

75. The protein of any one of claims 71-74, wherein the protein is attached to at least one payload.

76. The protein of claim 75, wherein the at least one payload comprises a small molecule, peptide, protein, nucleic acid, toxin, chemotherapeutic agent, liposome, nanoparticle, detectable label, or any useful combination thereof.

77. The protein of claim 76, wherein the detectable label comprises at least one magnetic label, fluorescent moiety, enzyme, light emitting particle, chemiluminescent probe, metal particle, non-metal colloidal particle, polymeric dye particle, pigment molecule, electrochemically active species, semiconductor nanocrystal, nanoparticle, quantum dot, gold particles, fluorophore, or radioactive label.

78. A method comprising contacting a cell with the protein of any one of claims 71-77.

79. A method of treating cancer in a patient comprising the administration of a zinc finger protein to the patient in need thereof wherein
i) the zinc finger protein binds to a cell surface extracellular DNA (exDNA) that is specific for a cancer cell and the zinc finger protein forms a zinc finger protein/exDNA complex with the exDNA;
ii) the zinc finger protein/exDNA complex is internalized into the cancer cell; and,
iii) the zinc finger protein/exDNA complex binds to an intracellular target.

80. The method of claim 79, wherein the zinc finger protein that binds a cell surface exDNA specific for a cancer cell is attached to an antibody that is internalized into the cancer cell and binds an intracellular target.

81. The method of claim 79, wherein the intracellular target is a DNA mutation specific for that cancer cell.

82. The method of claim 79, wherein the zinc finger protein that binds a cell surface exDNA specific for a cancer cell is attached to a CRISPR-derived sequence forming a zinc finger protein/CRISPR/exDNA complex to target specific intracellular DNA sequences, wherein the zinc finger protein/CRISPR/exDNA complex is internalized into the cancer cell.

83. The method of claim 79, wherein the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

84. A method of targeting or identifying cancer cells comprising using a zinc finger protein wherein
i) the zinc finger protein binds to a cell surface extracellular DNA (exDNA) that is specific for a cancer cell and the zinc finger protein forms a zinc finger protein/exDNA complex with the exDNA;
ii) the zinc finger protein/exDNA complex is internalized into the cancer cell; and,
iii) the zinc finger protein/exDNA complex binds to an intracellular target.

85. The method of claim 84, wherein the zinc finger protein that binds a cell surface exDNA specific for a cancer cell is attached to an antibody that is internalized into the cancer cell and binds an intracellular target, and wherein the antibody optionally is attached to a biomarker for visualization.

86. The method of claim 84, wherein the intracellular target is a DNA mutation specific for that cancer cell.

87. The method of claim 84, wherein the zinc finger protein that binds a cell surface exDNA specific for a cancer cell is attached to a CRISPR-derived sequence forming a zinc finger protein/CRISPR/exDNA complex to target specific intracellular DNA sequences, wherein the zinc finger protein/CRISPR/exDNA complex is internalized into the cancer cell.

88. The method of claim 84, wherein the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

89. The method of claim 84, wherein the cancer cells to be targeted or identified are in vivo in a patient suspected or diagnosed with cancer.

90. The method of claim 84, wherein the cancer cells to be targeted or identified are in an in vitro sample or biopsy from a patient suspected or diagnosed with cancer.

91. A method of inhibiting the growth of a cancer cell in a patient comprising the administration of a zinc finger protein to the patient in need thereof wherein
i) the zinc finger protein binds to a cell surface extracellular DNA (exDNA) that is specific for a cancer cell and the zinc finger protein forms a zinc finger protein/exDNA complex with the exDNA;
ii) the zinc finger protein/exDNA complex is internalized into the cancer cell; and,
iii) the zinc finger protein/exDNA complex binds to an intracellular target.

92. The method of claim 91, wherein the zinc finger protein that binds a cell surface exDNA specific for a cancer cell is attached to an antibody that is internalized into the cancer cell and binds an intracellular target.

93. The method of claim 91, wherein the intracellular target is a DNA mutation specific for that cancer cell.

94. The method of claim 91, wherein the zinc finger protein that binds a cell surface exDNA specific for a cancer cell is attached to a CRISPR-derived sequence forming a zinc finger protein/CRISPR/exDNA complex to target specific intracellular DNA sequences, wherein the zinc finger protein/CRISPR/exDNA complex is internalized into the cancer cell.

95. The method of claim 91, wherein the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

96. A method of diagnosing cancer in a patient comprising administration of a zinc finger protein to the patient suspected of having cancer, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the the biomarker is visualized or quantified to diagnose the cancer.

97. The method of claim 96, wherein the cancer cell is a precancerous cell.

98. The method of claim 97, wherein the zinc finger protein that binds a cell surface exDNA specific for a cancer cell is attached to an antibody, and wherein the antibody is attached to a biomarker for visualization.

99. A composition to treat cancer in a patient in need thereof comprising a zinc finger protein wherein
i) the zinc finger protein binds to a cell surface extracellular DNA (exDNA) that is specific for a cancer cell and the zinc finger protein forms a zinc finger protein/exDNA complex with the exDNA;
ii) the zinc finger protein/exDNA complex is internalized into the cancer cell; and,
iii) the zinc finger protein/exDNA complex binds to an intracellular target.

100. The composition of claim 99, wherein the zinc finger protein that binds a cell surface exDNA specific for a cancer cell is attached to an antibody that is internalized into the cancer cell and binds an intracellular target, and wherein the antibody optionally is attached to a biomarker for visualization.

101. The composition of claim 99, wherein the intracellular target is a DNA mutation specific for that cancer cell.

102. The composition of claim 99, wherein the zinc finger protein that binds a cell surface exDNA specific for a cancer cell is attached to a CRISPR-derived sequence forming a zinc finger protein/CRISPR/exDNA complex to target specific intracellular DNA sequences, wherein the zinc finger protein/CRISPR/exDNA complex is internalized into the cancer cell.

103. The composition of claim 99, wherein the zinc finger protein that binds a cell surface exDNA specific for a cancer cell is attached to a chemotherapeutic drug or prodrug forming a zinc finger protein/chemotherapeutic drug or prodrug/exDNA complex and wherein the zinc finger protein/chemotherapeutic drug or prodrug/exDNA complex is internalized into the cancer cell.

104. A composition for diagnosing cancer in a patient suspected of having cancer comprising a zinc finger protein, wherein the zinc finger protein has a biomarker attached and wherein the zinc finger protein binds to a cell surface extracellular DNA (exDNA) that is specific for a cancer cell, the zinc finger protein forms a zinc finger protein/exDNA complex with the exDNA, and wherein the biomarker is visualized or quantified to diagnose the cancer.

105. The composition of claim 104, wherein the cancer cell is a precancerous cell.

106. The composition of claim 104, wherein the cancer in a patient suspected of having cancer is diagnosed in vivo.

107. The composition of claim 104, wherein the cancer in a patient suspected of having cancer is diagnosed in an in vitro sample or biopsy from a patient suspected of having cancer.
